(19) United States
(12) Patent Application Publication
(10) Pub. No.: US 2010/0168421 A1
(43) Pub. Date: Jul. 1, 2010

(54) A NEW PEPTIDE DEFORMYLASE INHIBITOR COMPOUND AND MANUFACTURING PROCESS THEREOF

(75) Inventors: Jae Hoon KANG, Seoul (KR); Seung Woo YU, Suwon-si (KR); Hee Yeol LEE, Yongin-si (KR); Kyung Mi AN, Osan-si (KR); Bong Hwan CHO, Seongnam-si (KR)

Correspondence Address: NEW YORK PATENT GROUP, INC., 1215 AVE. N., SUITE 4-E, BROOKLYN, NY, 11230, US

(73) Assignee: ILDONG PHARMACEUTICAL CO., LTD., Seoul (KR)

(21) Appl. No.: 12/663,316

(22) PCT Filed: Jun. 4, 2008
(86) PCT No.: PCT/KR2008/003109

§ 371 Date: Dec. 8, 2009

(30) Foreign Application Priority Data
Jun. 7, 2007 (KR) 10-2007-0055482

(51) Int. Cl. C07D 413/12; C07D 401/12; C07D 405/12; C07D 211/60

(52) U.S. Cl. 544/130; 546/208; 546/214; 546/245

(57) ABSTRACT

The present invention relates to the novel antibacterial compounds having potent antibacterial activity as inhibitors of peptide deformylase. This invention further relates to pharmaceutically acceptable salts thereof, to processes for their preparation, and to pharmaceutical compositions containing them as an active ingredient.

### TECHNICAL FIELD

The present invention relates to the novel antibacterial compounds having potent antibacterial activity as inhibitors of peptide deformylase. This invention further relates to pharmaceutically acceptable salts thereof, to processes for their preparation, and to pharmaceutical compositions containing them as an active ingredient.

### BACKGROUND ART

Since Flemming's discovery of penicillin accidentally in 1920s, it has been developed for therapeutic injection in 1940s and from then on many of antibiotics have been developed systematically. Many classes of antibiotics have been produced such as β-lactams including penicillin and cephalosporin, aminoglycosides, phenyl-propanoids, tetracyclins, macrolides, glycopeptides, phosphonate, lipopeptides from natural products and quinolones, oxazolidinones from synthetic products. (Christopher T. Walsh et al., Chem. Review, 2005, 105, 391-395.).

But these antibiotics caused serious resistance to existing antibiotics. Recently published literature indicate that bacteria are rapidly acquiring resistance to well known antibiotics, including vancomycin and new agent such as linezolid (Staphylococcus aureus resistant to vancomycin—United States, 2002. MMWR 2002, 51(26), 565-567; linezolid resistance in a clinical isolate of Staphylococcus aureus. Lancet 2001, 358 (9277), 207-208). Therefore, there is an urgent need to discover antibiotics with new modes of action.

Most of antibiotics act by inhibiting one or more steps of bacterial protein biosynthesis. Although protein synthesizing process of bacteria and mammalian cells is similar overall, there is difference to allow for the selective blocking of this process in bacteria. One significant difference is the transformylation and subsequent deformylation of methionine (Richard J. White et al., Drug Discovery Today 2001, 6(18), 954-961).

Peptide deformylase (PDF) is unique metalloenzyme, which is utilizes a ferrous ion (Fe²⁺) to catalyze deformylation of N-formyl methionine (fMet, N-formylmethionine) in bacteria. In bacteria, protein synthesis starts with an N-formyl methionine (fMet), and as a result, all newly synthesized polypeptides carry a formylated N-terminus. PDF catalyzes the subsequent removal of the formyl group from the majority of those polypeptides, many of which undergo further N-terminal processing by methionine aminopeptidase (MAP) to produce mature proteins. Since protein synthesis in eukaryotic organisms does not depend on N-formyl methionine (fMet) for initiation, PDF inhibitors are expected to act as a new class of antimicrobial and antibacterial agents.

Numerous PDF inhibitors such as actinonine obtained from natural product have been structural feature; chelator+peptidomimetic.

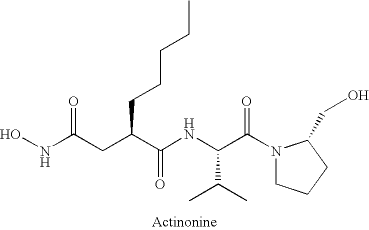

On the basis of the chelator structure, they can be classified into three different types: the thiols, the hydroxamic acids, and the N-formyl hydroxylamines.

Several PDF inhibitors have been reported in the literature some of which relevant are given here:

hydroxamic acid derivatives: WO 99/59568, WO 00/44373, WO 01/44178, WO 01/44179, WO 02/28829 and WO 02/081426

N-formyl hydroxylamines derivatives: WO 01/85160, WO 01/85170, WO 02/070540, WO 02/070541, WO 02/070653, WO 02/070654, WO 02/098901, WO 03/101442, WO 0035440, WO 99/39704, WO 00/35440, WO 00/58294, WO 00/61134, WO 01/10834, WO 01/10835, WO 03/089412 and WO 2004/033441

### DISCLOSURE OF INVENTION

### Technical Problem

Although a wide variety of compounds described in prior art have been developed as inhibitors of peptide deformylase, they did not result in a clinically useful compound. And PDF inhibitors are expected to block cross-resistance to the existing antibiotics.

In view of the rapid emergence of the multidrug-resistant bacteria, there is an urgent needed to develop antimicrobial and antibacterial agents with new modes of action.

The present invention fulfills this need.

### Technical Solution

The present invention relates to the novel hydroxamic acid and N-formyl hydroxylamine derivatives having potent antibacterial activity as inhibitors of peptide deformylase. This invention further relates to processes for their preparation, to intermediates useful in their preparation, and to pharmaceutical compositions containing them as an active ingredient:

The present invention relates to a compound of formula (I), all such racemic mixtures, optical isomers and diastereoisomers or a pharmaceutically acceptable salts thereof:

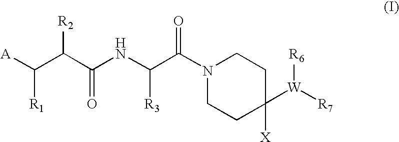

wherein, A is selected from the group of consisting of —C(═O)NHOH or —N(CHO)OH;

R₁ represents hydrogen, C₁₋₃ alkyl, C₄₋₆ cycloalkyl, halogen, or hydroxyl group;

R₂ represents hydrogen, straight or branched C″ alkyl, straight or branched C₂₋₆ alkenyl, C₄₋₆ cycloalkyl, C₄₋₆ heterocycle including nitrogen or oxygen, or benzyl group;

R₃ represents hydrogen, straight or branched C₁₋₆ alkyl, straight or branched C₂₋₆ alkenyl, C₄₋₆ cycloalkyl, phenyl or benzyl group;

X represents hydrogen or NR₄R₅;

Each of R₄ and R₅ is independently hydrogen, straight or branched C₁₋₃ alkyl, tert-butoxycarbonyl, benzyloxycarbonyl group;

W represents carbon or nitrogen;

Each of R₆ and R₇ is independently hydrogen, straight or branched C₁₋₃ alkyl, tert-butoxylcarbonyl, benzyloxycarbonyl, 2,2,2-trichloroethoxycarbonyl or a group of formula (IIa), or (IIb), or (IIc):

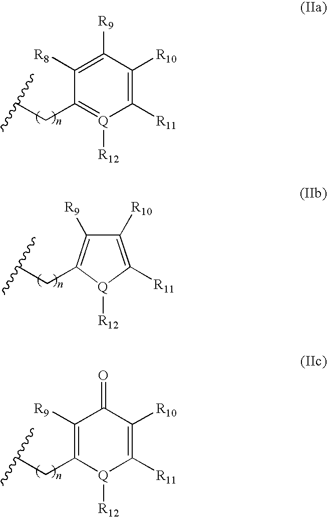

wherein, each of R₈, R₉, R₁₀, R₁₁ and R₁₂ is independently hydrogen, straight or branched C₁₋₃ alkyl, straight or branched C₁₋₃ alkylamine, C₃₋₆ cycloalkyl, C₄₋₆ heterocycle, C₁₋₃ alkoxyl, C₁₋₃ acyl, C₁₋₃ acyloxy, hydroxy, amido, halogen (fluoro, chloro, bromo and iodo), halogen-substituted C₁₋₃ alkyl, cyano, nitro or morpholinyl group;

Q represents carbon or nitrogen or oxygen;

n is 0 or 1 or 2.

### MODE FOR THE INVENTION

The present invention relates to the novel hydroxamic acid and N-formyl hydroxylamine derivatives having potent antibacterial activity as inhibitors of peptide deformylase. This invention further relates to processes for their preparation, to intermediates useful in their preparation, and to pharmaceutical compositions containing them as an active ingredient:

The present invention relates to a compound of formula (I), all such racemic mixtures, optical isomers and diastereoisomers or a pharmaceutically acceptable salts thereof:

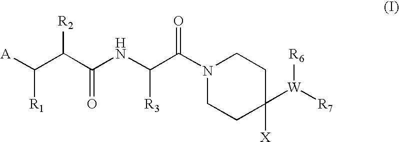

wherein, A is selected from the group of consisting of —C(═O)NHOH or —N(CHO)OH;

R₁ represents hydrogen, C₁₋₃ alkyl, C₄₋₆ cycloalkyl, halogen, or hydroxyl group;

R₂ represents hydrogen, straight or branched C₁₋₆ alkyl, straight or branched C₂₋₆ alkenyl, C₄₋₆ cycloalkyl, C₄₋₆ heterocycle including nitrogen or oxygen, or benzyl group;

R₃ represents hydrogen, straight or branched C₁₋₆ alkyl, straight or branched C₂₋₆ alkenyl, C₄₋₆ cycloalkyl, phenyl or benzyl group;

X represents hydrogen or NR₄R₅;

Each of R₄ and R₅ is independently hydrogen, straight or branched C₁₋₃ alkyl, tert-butoxycarbonyl, benzyloxycarbonyl group;

W represents carbon or nitrogen;

Each of R₆ and R₇ is independently hydrogen, straight or branched C₁₋₃ alkyl, tert-butoxylcarbonyl, benzyloxycarbonyl, 2,2,2-trichloroethoxycarbonyl or a group of formula (IIa), or (IIb), or (IIc):

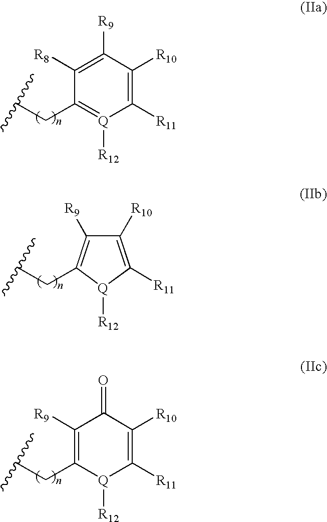

wherein, each of R₈, R₉, R₁₀, R₁₁ and R₁₂ is independently hydrogen, straight or branched C₁₋₃ alkyl, straight or branched C₁₋₃ alkylamine, C₃₋₆ cycloalkyl, C₄₋₆ heterocycle, C₁₋₃ alkoxyl, C₁₋₃ acyl, C₁₋₃ acyloxy, hydroxy, amido, halogen (fluoro, chloro, bromo and iodo), halogen-substituted C₁₋₃ alkyl, cyano, nitro or morpholinyl group;

Q represents carbon or nitrogen or oxygen;

n is 0 or 1 or 2.

The compounds of this invention may possess one or more asymmetric centers because of the presence of asymmetric carbon atoms. Therefore, the invention includes all such racemic mixtures, optical isomers and diastereoisomers thereof.

Compounds of the invention may be administered in pharmaceutically acceptable salt forms, hydrate forms or solvate forms. Such salts include acid addition salts, formed with hydrochloric acid, hydrobromic acid, sulfuric acid, nitric acid, methane-sulfonic acid, p-toluenesulfonic acid, phosphoric acid, acetic acid, pyruvic acid, citric acid, succinic acid, lactic acid, tartaric acid, fumaric acid, maleic acid, stearic acid and salicylic acid. Salts may also be formed with sodium, potassium, magnesium and calcium salts.

The present invention provides a process for preparing of formula (I), or pharmaceutically acceptable salt, hydrate or solvate thereof.

A compound of invention wherein A is —C(═O)NHOH group may be prepared by reacting a compound of formula (III) with hydroxylamine or N- and/or O-protected hydroxylamine, and thereafter removing any N- or O-protecting groups:

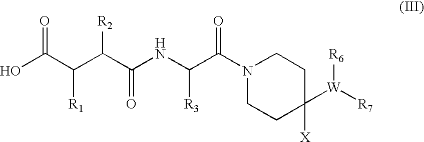

wherein, R₁, R₂, R₃, R₆, R₇, W and X are the same as defined above.

Reaction of formula (III) with hydroxylamine or N- and/or O-protected hydroxylamine may be carried out according to standard peptide coupling conditions. The reactions are typically carried out in the presence of coupling reagents (e.g. pentafluorophenol, N,O-demethylhydroxylamine, DMAP/ECCI, EDCI/HOBt/NMM, etc.) in an appropriate solvent (e.g. tetrahydrofuran, dichloromethane, N,N-demethylformamide, etc.). Deprotection of benzyl group may be carried out in the presence of hydrogenation catalyst, preferably a palladium catalyst (e.g. palladium on carbon or palladium black). The reaction can be achieved under a hydrogen atmosphere for about 2 to about 24 hours.

Deprotection of tert-butyl group may be carried out in the presence of an appropriate acid, such as hydrochloric acid or trifluoroacetic acid. The reaction can be achieved by stirring for about 1 to about 24 hours.

Compound of formula (III) may be prepared by reacting a compound of formula (IV) with a compound of formula (Va) (or Vb, or Vc) or salt thereof.

Reaction of formula (IV) with a compound of formula (Va) (or Vb, or Vc) or salt thereof may be carried out according to the standard peptide coupling conditions.

The reaction is typically carried out in the presence of a coupling reagent (e.g. pentafluorophenol, N,O-dimethylhydroxylamine, DMAP/EDCI, EDCI/HOBt/NMM, etc.), in an appropriate solvent (e.g. tetrahydrofuran, dichloromethane, N,N-dimethylformamide, etc.):

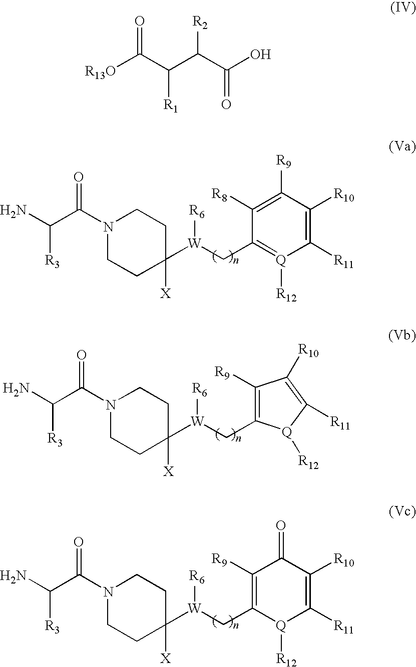

wherein R₁, R₂, R₃, R₆, R₈, R₉, R₁₀, R₁₁, R₁₂, Q, W, X and n are the same as defined above and R₁₃ is a hydroxy protecting group, such as methyl, ethyl, tert-butyl, and benzyl group.

Carboxylic acids of formula (IV) may be prepared according to any of a variety of methods described in the literature.

Also, compound of the invention wherein A is —N(CHO)OH group may be prepared by reacting a compound of formula (VI) with a compound of formula (Va) (or Vb, or Vc) or salt thereof:

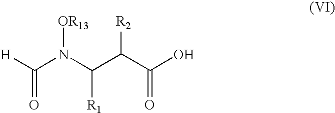

Reaction of formula (VI) with a compound of formula (Va) (or Vb, or Vc) or salt thereof may be carried out according to the standard peptide coupling conditions.

The reaction is typically carried out in the presence of a coupling reagent (e.g. pentafluorophenol, N,O-dimethylhydroxylamine, DMAP/EDCI, EDCI/HOBt/NMM, etc.), in an appropriate solvent (e.g. tetrahydrofuran, dichloromethane, N,N-dimethylformamide, etc.). Deprotection of benzyl group may be carried out in the presence of the hydrogenation catalyst, preferably a palladium catalyst (e.g. palladium on carbon or palladium black). The reaction can be achieved under a hydrogen atmosphere for about 2 to about 24 hours.

Deprotection of tert-butoxycarbonyl group may be carried out in the presence of an appropriate acid, such as hydrochloric acid or trifluoroacetic acid. The reaction can be achieved by stirring for about 1 to about 24 hours:

Wherein, R₁, R₂ and R₁₃ are the same as defined above.

Carboxylic acids of formula (VI) may be prepared according to any of a variety of methods described in the literature

The compound of formula (Va) (or Vb, or Vc) or salt thereof may be obtained by reacting a compound of formula (VII) with a compound of formula (VIIIa) (or VIIIb, or VIIIc) or salt thereof.

The reaction is typically carried out in the presence of a coupling reagent (e.g. pentafluorophenol, N,O-dimethylhydroxylamine, DMAP/EDCI, EDCI/HOBt/NMM, etc.), in an appropriate solvent (e.g. tetrahydrofuran, dichloromethane, N,N-dimethylformamide, etc.):

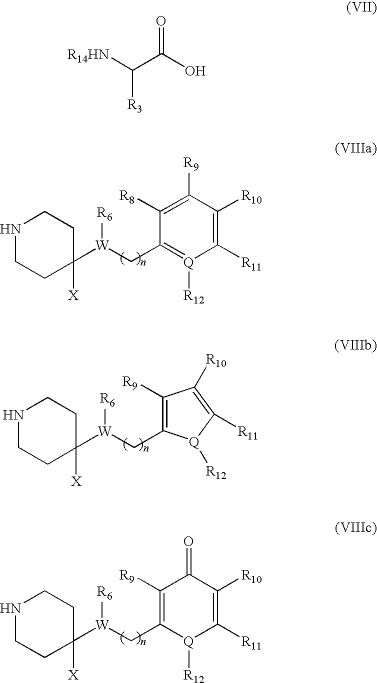

wherein, R₃, R₆, R₈, R₉, R₁₀, R₁₁, R₁₂, Q, W, X and n are the same as defined above and R₁₄ is a amino protecting group, such as tert-butoxycarbonyl, benzyloxycarbonyl or triphenylmethyl group.

Carboxylic acids of formula (VII) may be prepared according to any of a variety of methods described in the literature.

Compound of formula (VIIIa) wherein X is hydrogen, W is nitrogen, n is 0, may be obtained by reacting compound of formula (X) with compound of formula (XIa) wherein Y is amine, in haloalkane solvent for about 4 to about 24 hours. Thereafter, the reaction can be achieved by using reducing agents, preferably sodium borohydride or sodium cyanoborohydride or sodium triacetoxyborohydride for about 2 to about 24 hours.

Compound of formula (VIIIa) wherein X is protected amine, W is carbon, n is 0, may be obtained by reacting compound of formula (IX) wherein W is halomethyl or salt thereof with Grignard reagent formed from compound of formula (XIa) wherein Y is halogen.

Compound of formula (VIIIa) (or VIIIb, or VIIIc) wherein X is hydrogen, W is nitrogen, n is 1, may be obtained by compound of formula (IX) wherein W is amine or salt thereof with compound of formula (XIa) wherein Y is formyl(—CHO), in alcohol solvent for about 4 to about 24 hours. Thereafter the reaction can be achieved by using reducing agents, preferably sodium borohydride or sodium cyanoborohydride or sodium triacetoxyborohydride for about 2 to about 24 hours.

As another method, compound of formula (VIIIa) (or VIIIb, or VIIIc) wherein X is hydrogen, W is nitrogen, n is 1 or 2, may be obtained by reacting compound of formula (IX) or salt thereof with compound of formula (XIa) (or XIb, or XIc) wherein Y is halomethyl or haloethyl. The reaction is typically carried out in the presence of an appropriate base (e.g. triethylamine, N,N-diisopropylethylamine, potassium carbonate, etc) at 0˜100° C. for about 2 to about 24 hours:

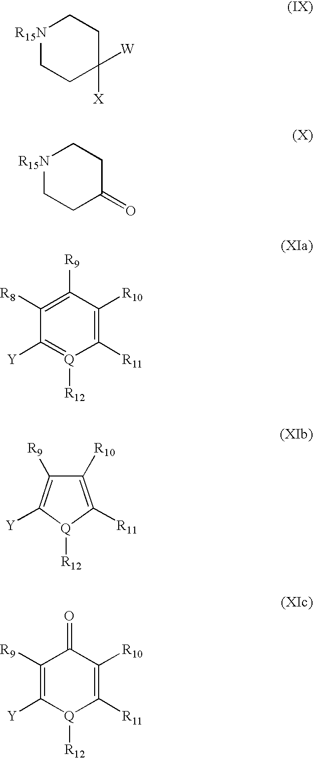

wherein, R₈, R₉, R₁₀, R₁₁, R₁₂, Q, W and X are the same as defined above and Y is amine, formyl, bromo, halomethyl, haloethyl group and R₁₅ is a amino protecting group, such as tert-butoxycarbonyl, benzyloxycarbonyl group.

Formula (IX) may be prepared according to any of a variety of methods described in the literature.

The examples which follow illustrate embodiments of the invention but are not intended to limit the scope in any way.

### General Procedure I

### Synthesis of [1-((S)-2-Amino-3,3-dimethyl-butyryl)-piperidin-4-yl]-(4-methyl-benzyl)-carbamic acid benzyl ester hydrochloride (Scheme 1)

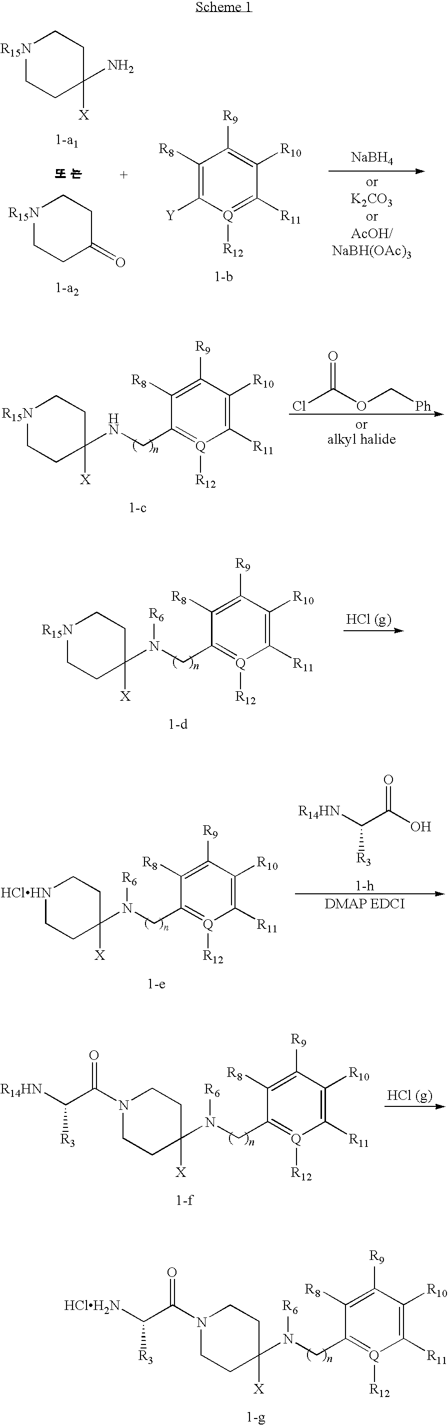

### Step 1-1: 4-(4-Methyl-benzylamino)-piperidine-1-carboxylic acid tert-butyl ester (1-c, n=1)

To a solution of compound 1-a₁ (R₁₅=tert-butoxycarbonyl, X═H, 2.50 g, 12.48 mmol) in ethanol (100 mL) was added compound 1-b (R₈═R₉═R₁₁═R₁₂═H, R₁₀=Me, Q=C, Y═CHO, 1.55 g, 12.48 mmol). The reaction mixture was refluxed for 4 hours before adding sodium borohydride (0.52 g, 13.73 mmol, 1.10 eq.) and stirred at room temperature for 24 hours. The reaction mixture was diluted with H₂O and extracted with ethyl acetate. The organic layer was washed with H₂O and brine and dried over magnesium sulfate. Solvent was concentrated in vacuo to give the title compound as a pale yellow oil which was used in next step without further purification (3.79 g, 100%).

¹H-NMR (CDCl₃): δ 7.20-7.22 (m, 2H), 7.12-7.14 (m, 2H), 4.02 (bs, 2H), 3.78 (s, 2H), 2.73-2.78 (m, 2H), 2.62-2.60 (m, 1H), 2.33 (s, 3H), 1.80-1.90 (m, 2H), 1.45 (s, 9H), 1.18-1.38 (m, 2H).

### Step 1-2: 4-(4-Methoxycarbonyl-benzylamino)-piperidine-1-carboxylic acid tert-butyl ester (1-c, n=1)

To a solution of compound 1-a₁ (R₁₅=tert-butoxycarbonyl, X═H, 1.00 g, 4.99 mmol) in acetonitrile (50 mL) was added compound 1-b (R₈═R₉═R₁₁═R₁₂═H, R₁₀═C(═O)OMe, Q=C, Y=bromomethyl, 1.37 g, 5.99 mmol, 1.20 eq.) and potassium carbonate (1.04 g, 7.49 mmol, 1.50 eq.). The reaction mixture was stirred at room temperature for 18 hours. Potassium carbonate was removed by filtration and filtrate was concentrated in vacuo, the residue was purified by column chromatography to give the title compound as a pale yellow solid (1.30 g, 75%).

¹H-NMR (CDCl₃): δ 7.99-8.01 (m, 2H), 7.40-7.42 (m, 2H), 4.09 (bs, 2H), 3.91 (s, 3H), 3.89 (s, 2H), 2.74-2.84 (m, 2H), 2.60-2.70 (m, 1H), 1.81-1.90 (m, 2H), 1.45 (s, 9H), 1.24-1.37 (m, 2H).

### Step 1-3: 4-Phenylamino-piperidine-1-carboxylic acid tert-butyl ester (1-c, n=0)

To a solution of compound 1-a₂ (R₁₅=tert-butoxycarbonyl, 3.00 g, 15.06 mmol) in dichloroethane (30 mL) was added compound 1-b (R₈═R₉═R₁₀═R₁₁═R₁₂═H, Q=C, Y═NH₂, 1.54 ml, 1690 mmol, 1.12 eq.) and acetic acid (1.02 ml, 17.82 mmol, 1.18 eq.). After sodium triacetoxyborohydride was added and stirred at room temperature for 20 hours. The reaction mixture was adjusted to pH 10 with 2 N sodium hydroxide solution and extracted with dichloromethane. The organic layer was dried over magnesium sulfate and concentrated in vacuo. The residue was purified by cyclohexane and gave the title compound as white solid (2.70 g, 65%).

¹H-NMR (DMSO-d₆): δ 7.03-7.07 (m, 2H), 6.56-6.59 (m, 2H), 6.50 (t, J=7.2 Hz, 1H), 3.85-3.91 (m, 2H), 3.37-3.42 (m, 1H), 2.90 (bs, 2H), 1.85-1.89 (m, 2H), 1.40 (s, 9H), 1.16-1.26 (m, 2H).

### Step 2-1: 4-[Benzyloxycarbonyl-(4-methyl-benzyl)-amino]-piperidine-1-carboxylic acid tert butyl ester (1-d, n=1, R₆=benzyloxycarbonyl)

To a solution of compound 1-c (n=1, 3.77 g, 12.38 mmol) in THF (50 mL) and H₂O (50 mL) was added aqueous sodium hydroxide (15 ml, 4.00 eq.) and cooled to 0° C. After benzylchloro formate (3.18 ml, 22.29 mmol, 1.80 eq.) was slowly added and stirred at room temperature for 20 hours. The reaction mixture was diluted with H₂O and extracted with ethyl acetate. The organic layer was washed with H₂O and dried over magnesium sulfate. Solvent was evaporated under reduced pressure and residue was purified by column chromatography to give the title compound as a colorless oil (4.82 g, 89%).

¹H-NMR (CDCl₃): δ 7.35-7.39 (m, 5H), 7.25-7.30 (m, 2H), 7.07-7.10 (m, 2H), 5.13-5.28 (m, 2H), 4.68 (s, 2H), 4.38-4.48 (m, 2H), 4.09-4.14 (m, 3H), 2.55-2.76 (m, 2H), 2.32 (s, 3H), 1.55-1.66 (m, 2H), 1.42 (s, 9H).

### Step 2-2: 4-(Methyl-phenyl-amino)-piperidine-1-carboxylic acid tert-butyl ester (1-d, n=0, R₆=Me)

To a solution of compound 1-c (n=0, 3.04 g, 11.00 mmol) in DMF (55 mL) was added iodomethane (5.20 ml, 83.53 mmol, 7.60 eq.) and potassium carbonate (11.55 g, 83.56 mmol, 7.60 eq.). After it was stirred at room temperature for 18 hours, the reaction mixture was diluted with H₂O and extracted with ethyl acetate. The organic layer was washed with H₂O and dried over magnesium sulfate. Solvent was evaporated under reduced pressure and residue was purified by column chromatography to give the title compound as a white solid (1.20 g, 38%).

¹H-NMR (CDCl₃): δ 7.22-7.27 (m, 2H), 6.81 (d, J=8.4 Hz, 2H), 6.73 (t, J=7.4 Hz, 1H), 4.23 (bs, 2H), 3.67-3.74 (m, 1H), 2.76-2.81 (m, 5H), 1.62-1.74 (m, 4H), 1.47 (s, 9H).

### Step 3: (4-Methyl-benzyl)-piperidin-4-yl-carbamic acid benzyl ester hydrochloride (1-e)

Compound 1-d (4.82 g, 10.99 mmol) was dissolved in ethyl acetate (60 mL) and saturated with gaseous hydrochloric acid, and the reaction mixture stirred until all of the starting material was consumed. The mixture was concentrated to give amine hydrochloride salt as a white crystalline solid which was used in next step without further purification (4.10 g, 100%).

¹H-NMR (D₂O): δ 6.68-6.85 (m, 9H), 4.64 (s, 2H), 4.10 (s, 2H), 3.77-3.86 (m, 1H), 3.11-3.21 (m, 2H), 2.67-2.80 (m, 2H), 1.85 (s, 3H), 1.69-1.82 (m, 2H), 1.41-1.49 (m, 2H).

### Step 4: [1-((S)-2-tert-Butoxycarbonylamino-3,3-dimethyl-butyryl)-piperidin-4-yl]-(4-methyl-benzyl)-carbamic acid benzyl ester (1-f)

To a solution of compound 1-h (R₃=tert-butyl, R₁₄=tert-butoxylcarbonyl, 1.34 g, 5.78 mmol) in dichloromethane was added compound 1-e (2.60 g, 6.94 mmol, 1.20 eq.), 4-dimethylaminopyridine (DMAP) (1.77 g, 14.45 mmol, 2.50 eq.) and 1-ethyl-3-(3-dimethylaminopropyl)carbodiimide hydrochloride (EDCI) (1.33 g, 6.94 mmol, 1.20 eq.). The mixture was stirred at room temperature for 18 hours. The reaction mixture was washed with aqueous 1 M potassium hydrogen sulfate, aqueous saturated sodium bicarbonate and brine. The organic layer was dried over magnesium sulfate and concentrated in vacuo. The residue was purified by column chromatography to give the title compound as a white solid (2.94 g, 92%).

¹H-NMR (CDCl₃): δ 7.20-7.40 (m, 5H), 7.00-7.15 (m, 4H), 5.28-5.32 (m, 1H), 5.10-5.24 (m, 2H), 4.66-4.69 (d, J=12.4 Hz, 1H), 4.30-4.49 (m, 3H), 4.05-4.16 (m, 1H), 2.95-3.10 (m, 1H), 2.40-2.59 (m, 1H), 2.31 (s, 3H), 1.50-1.78 (m, 4H), 1.43 (s, 9H), 0.93 (s, 9H).

### Step 5: [1-((S))-2-Amino-3,3-dimethyl-butyryl]-piperidin-4-yl]-(4-methyl-benzyl)-carbamic acid benzyl ester hydrochloride (1-g)

Compound 1-f (2.94 g, 5.33 mmol) was dissolved in ethyl acetate (50 mL) and saturated with gaseous hydrochloric acid, and the reaction mixture stirred until all of the starting material was consumed. The mixture was concentrated to give amine hydrochloride salt as a white crystalline solid which was used in next step without further purification (2.55 g, 98%).

¹H-NMR (CD₃OD): δ 7.18-7.48 (m, 5H), 7.08 (s, 4H), 5.10-5.38 (m, 2H), 4.55-4.63 (m, 1H), 4.47 (s, 2H), 4.07-4.15 (m, 2H), 3.08-3.18 (m, 2H), 2.62-2.71 (m, 1H), 2.29 (s, 3H), 1.65-1.90 (m, 4H), 1.04 (s, 9H).

### General Procedure II

### Synthesis of [1-((S)-2-Amino-3,3-dimethyl-butyryl)-4-(4-fluoro-benzyl)-piperidin-4-yl]-carbamic acid benzyl ester hydrochloride (Scheme 2)

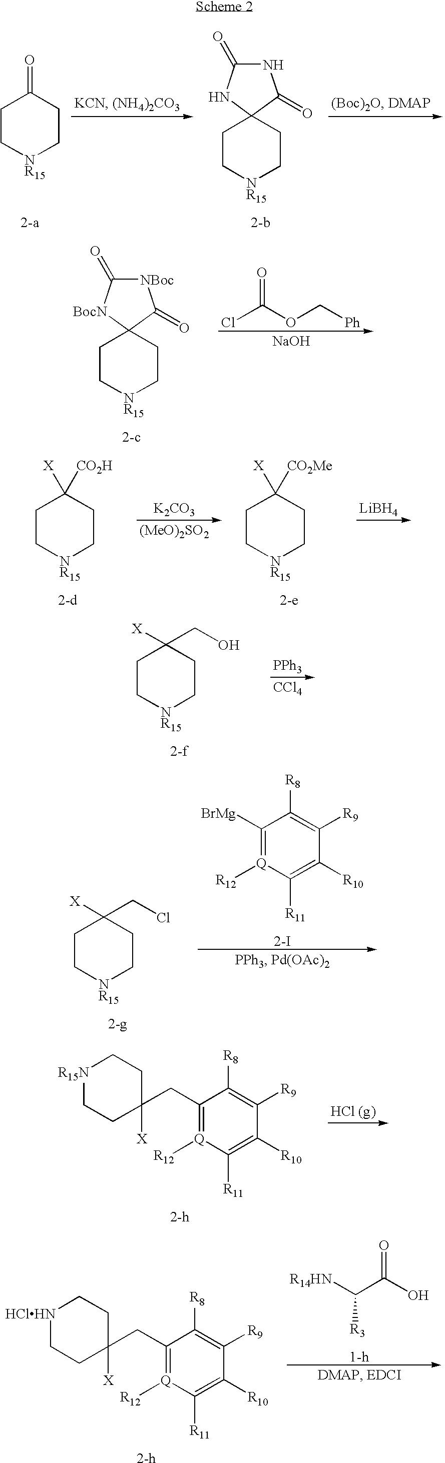

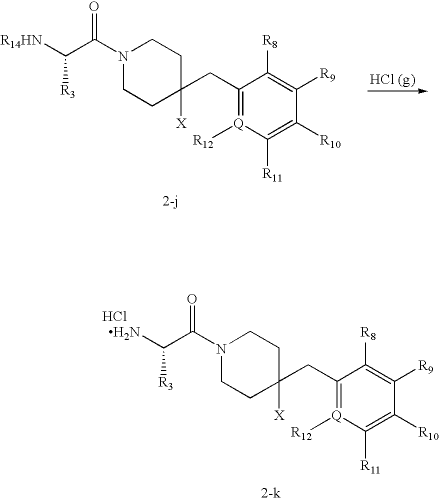

### Step 1: 2,4-Dioxo-1,3,8-triaza-spiro[4,5]decane-8-carboxylic acid tert-butyl ester (2-b)

To a solution of compound 2-a (R₁₅=tert-butoxycarbonyl, 10.00 g, 50.19 mmol) in methanol (190 mL) was added potassium cyanide (10.13 g, 155.58 mmol, 3.10 eq.) and aqueous ammonium carbonate (14.14 g, 150.56 mmol, 3.00 eq.) solution (190 mL). After reaction mixture was refluxed for 20 hours, methanol was evaporated and recrystallized from H₂O gave a pale yellow solid which was used in next step without further purification (8.40 g, 62%).

¹H-NMR (DMSO-d₆): δ 10.73 (s, 1H), 8.54 (s, 1H), 3.76-3.87 (m, 2H), 3.00-3.20 (bs, 2H), 1.62-1.72 (m, 2H), 1.48-1.58 (m, 2H), 1.40 (s, 9H).

### Step 2: 2,4-Dioxo-1,3,8-triaza-spiro[4,5]decane-1,3,8-tricarboxylic acid tri-tert butyl ester (2-c)

A solution of compound 2-h (8.34 g, 30.97 mmol) in THF (200 mL) was cooled to 0° C. Di-tert-butyl dicarbonate (1690 g, 77.42 mmol, 2.50 eq.) and 4-dimethylaminopyridine (DMAP) (0.096 g, 0.78 mmol, 0.025 eq.) was slowly added and stirred at room temperature for 18 hours. After reaction mixture was concentrated to dryness, the residue was treated with dichloromethane and washed with 2 N hydrochloric acid, aqueous saturated sodium bicarbonate and brine. The organic layer was dried over magnesium sulfate and concentrated in vacuo to give the title compound as a white solid which was used in next step without further purification (14.54 g, 100%).

¹H-NMR (CDCl₃): δ 4.00-4.28 (m, 2H), 3.30-3.52 (m, 2H), 2.61-2.73 (m, 2H), 1.70-1.80 (m, 2H), 1.59 (s, 9H), 1.55 (s, 9H), 1.47 (s, 9H).

### Step 3: 4-Benzyloxycarbonylamino-piperidine-1,4-dicarboxylic acid mono-tert-butyl ester (2-d, X=NHCbz)

To the solution of compound 2-c (0.50 g, 2.05 mmol) in THF (4 mL) was added aqueous sodium hydroxide (0.17 g, 4.30 mmol, 2.10 eq.) solution (4 mL). The reaction mixture was cooled to 0° C. Benzyl chloroformate (0.32 ml, 2.25 mmol, 1.10 eq.) was slowly added and stirred at room temperature for 24 hours. After the reaction mixture was washed with diethyl ether, aqueous layer was adjusted to pH 2.5 with 1 N hydrochloric acid and extracted with ethyl acetate. The organic layer was dried over magnesium sulfate and concentrated in vacuo to give the title compound as a white solid which was used in next step without further purification (0.55 g, 71%).

¹H-NMR (CDCl₃): δ 7.29-7.39 (m, 5H), 5.10 (s, 2H), 3.84 (bs, 2H), 3.05-3.15 (m, 2H), 1.95-2.12 (m, 4H), 1.45 (s, 9H).

### Step 4: 4-Benzyloxycarbonylamino-piperidine-1,4-dicarboxylic acid 1-tert-butyl ester 4-methyl ester (2-e)

To a solution of compound 2-d (0.96 g, 2.54 mmol) in acetone (50 mL) was added potassium carbonate (0.70 g, 5.07 mmol, 2.00 eq.) and dimethyl sulfate (0.26 ml, 2.79 mmol, 1.10 eq.). After refluxed for 1 hour, potassium carbonate was removed by filtration and acetone was evaporated to dryness. The residue was diluted with ethyl acetate and washed with aqueous saturated sodium bicarbonate and brine. The organic layer was dried over magnesium sulfate and concentrated in vacuo to give the title compound as a pale yellow oil which was used in next step without further purification (0.99 g, 99%).

¹H-NMR (CDCl₃): δ 7.29-7.40 (m, 5H), 5.09 (s, 2H), 3.83 (bs, 2H), 3.70 (s, 3H), 3.04-3.15 (m, 2H), 1.92-2.10 (m, 4H), 1.45 (s, 9H).

### Step 5: 4-Benzyloxycarbonylamino-4-hydroxymethyl-piperidine-1-carboxylic acid tert-butyl ester (2-f)

To a solution of compound 2-e (1.50 g, 3.82 mmol) in THF (50 mL) was added lithium borohydride (0.10 g, 4.59 mmol, 1.20 eq.) and stirred at room temperature for 20 hours. The reaction mixture was diluted with H₂O and extracted with ethyl acetate. The organic layer was dried over magnesium sulfate and concentrated in vacuo. The residue was purified by column chromatography to give the title compound as a white solid (1.20 g, 86%).

¹H-NMR (CDCl₃): δ 7.28-7.44 (m, 5H), 5.11 (s, 2H), 3.85 (bs, 2H), 3.75 (s, 2H), 3.02-3.13 (m, 2H), 1.92-2.13 (m, 4H), 1.44 (s, 9H).

### Step 6: 4-Benzyloxycarbonylamino-4-chloromethyl-piperidine-1-carboxylic acid tert-butyl ester (2-g)

To a solution of compound 2-f (1.00 g, 2.74 mmol) in benzene (20 mL) was added carbon tetrachloride (15 mL) and triphenylphosphine (1.08 g, 4.12 mmol, 1.50 eq.) and then refluxed for 4 hours. After the white solid was removed by filtration and filtrate was evaporated to dryness. The residue was purified by column chromatography to give the title compound as a pale yellow oil (0.76 g, 72%).

¹H-NMR (CDCl₃): δ 7.24-7.42 (m, 5H), 5.10 (s, 2H), 3.83 (bs, 2H), 3.55 (s, 2H), 3.02-3.11 (m, 2H), 1.91-2.13 (m, 4H), 1.44 (s, 9H).

### Step 7: 4-Benzyloxycarbonylamino-4-(4-fluoro-benzyl)-piperidine-1-carboxylic acid tert-butyl ester (2-h)

To a solution of compound 2-g (0.70 g, 1.83 mmol) in THF (20 mL) was added triphenylphosphine (0.58 g, 2.19 mmol, 1.20 eq.) and palladium (II) acetate (0.041 g, 0.18 mmol, 0.10 eq.). After the reaction mixture was cooled to 0° C., 2-1 (R₈═R₉═R₁₁═R₁₂═H, R₁₀═F, Q=C, 3.66 ml, 3.66 mmol, 2.00 eq.) in THF solution was slowly added and stirred at room temperature for 20 hours. The reaction mixture was diluted with H₂O and extracted with ethyl acetate. The organic layer was dried over magnesium sulfate and concentrated in vacuo. The residue was purified by column chromatography to give the title compound as a pale yellow oil (0.57 g, 70%).

¹H-NMR (CDCl₃): δ 7.20-7.45 (m, 7H), 6.90-7.01 (m, 2H), 5.25 (s, 2H), 3.74-3.81 (m, 2H), 3.22-3.25 (m, 2H), 2.66 (s, 2H), 2.13-2.20 (m, 2H), 1.89-1.95 (m, 2H), 1.45 (s, 9H).

### Step 8: [4-(4-Fluoro-benzyl)-piperidin-4-yl]-carbamic acid benzyl ester hydrochloride (24)

Compound 2-h (1.20 g, 2.71 mmol) was dissolved in ethyl acetate (30 mL) and saturated with gaseous hydrochloric acid, and the reaction mixture stirred until all of the starting material was consumed. The mixture was concentrated to give amine hydrochloride salt as a white crystalline solid which was used in next step without further purification (0.98 g, 95%).

¹H-NMR (D₂O): δ 6.79-7.10 (m, 7H), 6.60-6.78 (m, 2H), 4.70 (s, 2H), 4.10-4.28 (m, 2H), 3.19-3.30 (m, 2H), 2.53 (s, 2H), 2.11-2.22 (m, 2H), 1.90-1.99 (m, 2H).

### Step 9: [1-((S)-2-tert-Butoxycarbonylamino-3,3-dimethyl-butyryl)-4-(4-fluoro-benzyl)-piperidin-4-yl]-carbamic acid benzyl ester (2-j)

To a solution of compound 1-h (R₃=tert-butyl, R₁₄=tert-butoxycarbonyl, 0.47 g, 2.03 mmol) in dichloromethane (30 mL) was added compound 24 (0.92 g, 2.43 mmol, 1.20 eq.), 4-dimethylaminopyridine (DMAP) (0.62 g, 5.07 mmol, 2.50 eq.) and 1-ethyl-3-(3-dimethylaminopropyl)carbodiimide hydrochloride (EDCI) (0.47 g, 2.43 mmol, 1.20 eq.). The mixture was stirred at room temperature for 18 hours. The reaction mixture was washed with aqueous 1 M potassium hydrogen sulfate, aqueous saturated sodium bicarbonate and brine. The organic layer was dried over magnesium sulfate and concentrated in vacuo. The residue was purified by column chromatography to give the title compound as a white solid (1.04 g, 92%).

¹H-NMR (CDCl₃): δ 7.22-7.46 (m, 7H), 6.89-7.01 (m, 2H), 5.27-5.35 (m, 1H), 5.23 (s, 2H), 3.75-3.85 (m, 2H), 3.24-3.29 (m, 2H), 2.68 (s, 2H), 2.13-2.25 (m, 2H), 1.90-1.98 (m, 2H), 1.45 (s, 9H), 0.89-1.01 (s, 9H).

### Step 10: [1-((S)-2-Amino-3,3-dimethyl-butyryl)-4-(4-fluoro-benzyl)-piperidin-4-yl]-carbamic acid benzyl ester hydrochloride (2-k)

Compound 2-j (1.00 g, 1.80 mmol) was dissolved in ethyl acetate (30 mL) and saturated with gaseous hydrochloric acid, and the reaction mixture stirred until all of the starting material was consumed. The mixture was concentrated to give amine hydrochloride salt as a white crystalline solid which was used in next step without further purification (0.88 g, 99%).

¹H-NMR (D₂O): δ 6.72-7.15 (m, 7H), 6.50-6.70 (m, 2H), 4.81-4.99 (m, 1H), 4.65-4.80 (m, 2H), 3.65-3.70 (m, 2H), 3.15-3.20 (m, 2H), 2.40 (s, 2H), 2.11-2.20 (m, 2H), 1.85-1.95 (m, 2H), 0.78-0.90 (s, 9H).

### General Procedure III

### Synthesis of [1-((S)-2-Amino-3,3-dimethyl-butyryl)-piperidin-4-yl]-furan-2-ylmethyl-carbamic acid benzyl ester hydrochloride (Scheme 3)

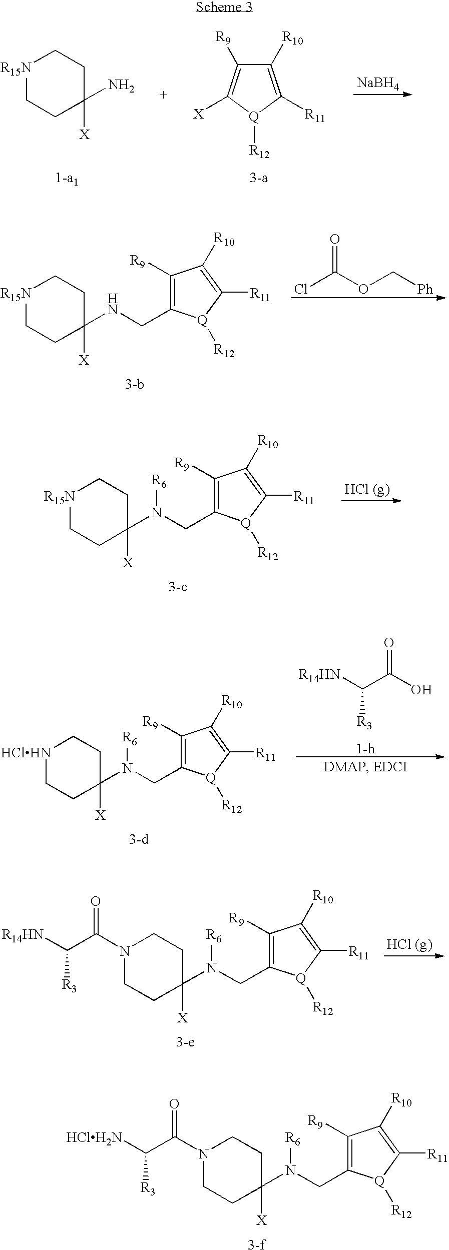

### Step 1: 4-[(Furan-2-ylmethyl)-amino]-piperidine-1-carboxylic acid tert-butyl ester (3-b)

To a solution of compound 1-a₁ (R₁₅=tert-butoxycarbonyl, X═H, 4.00 g, 19.97 mmol) in ethanol (150 mL) was added 3-a (R₉═R₁₀═R₁₁═H, Q=0, Y═CHO, 1.92 g, 19.97 mmol). The reaction mixture was reflux for 4 hours before adding sodium borohydride (0.83 g, 21.97 mmol, 1.10 eq.) and further stirred at room temperature for 24 hours. The reaction mixture was diluted with H₂O and extracted with ethyl acetate. The organic layer was washed with H₂O, brine and dried over magnesium sulfate. The mixture was concentrated in vacuo to give the title compound as a pale yellow oil which was used in next step without further purification (5.38 g, 96%).

¹H-NMR (CDCl₃): δ 7.33-7.37 (m, 1H), 6.29-6.30 (m, 1H), 6.15-6.19 (m, 1H), 4.03 (bs, 2H), 3.82 (s, 2H), 2.72-2.85 (m, 2H), 2.58-2.67 (m, 1H), 1.78-1.87 (m, 2H), 1.45 (s, 9H), 1.22-1.31 (m, 2H).

### Step 2: 4-(Benzyloxycarbonyl-furan-2-ylmethyl-amino)-piperidine-1-carboxylic acid tert-butyl ester (3-c, R₆=benzyloxycarbonyl)

To a solution of compound 3-h (5.30 g, 18.90 mmol) in THF (60 mL) and H₂O (60 mL) was added aqueous sodium hydroxide (20 ml, 4.00 eq.). The reaction mixture was cooled to 0° C. After benzylchloro formate (4.86 ml, 34.03 mmol, 1.80 eq.) was slowly added and stirred at room temperature for 20 hours. The reaction mixture was diluted with H₂O and extracted with ethyl acetate. The organic layer was washed with H₂O and dried over magnesium sulfate. Solvent was evaporated under reduced pressure and residue was purified by column chromatography to give the title compound as a pale yellow (5.96 g, 76%).

¹H-NMR (CDCl₃): δ 7.29-7.37 (m, 7H), 6.26-6.29 (m, 1H), 5.16 (s, 2H), 4.60 (s, 1H), 4.37 (s, 2H), 4.14 (bs, 2H), 2.70 (bs, 2H), 1.55-1.70 (m, 4H), 1.45 (s, 9H).

### Step 3: Furan-2-ylmethyl-piperidin-4-yl-carbamic acid benzyl ester hydrochloride (3-d)

Compound 3-c (5.96 g, 14.38 mmol) was dissolved in ethyl acetate (60 mL) and saturated with gaseous hydrochloric acid, and the reaction mixture stirred until all of the starting material was consumed. The mixture was concentrated to give amine hydrochloride salt as a white crystalline solid which was used in next step without further purification (5.00 g, 99%).

¹H-NMR (D₂O): δ 7.05-7.30 (m, 7H), 6.70-6.88 (m, 1H), 4.93 (s, 2H), 4.21-4.30 (m, 2H), 3.81-3.92 (m, 1H), 3.23-3.32 (m, 2H), 2.78-2.90 (m, 2H), 1.65-1.75 (m, 2H), 1.38-1.45 (m, 2H).

### Step 4: [1-((S)-2-tert-Butoxycarbonylamino-3,3-dimethyl-butyryl)-piperidin-4-yl]-furan-2-ylmethyl-carbamic acid benzyl ester (3-e)

To a solution of compound 1-h (R₃=tert-butyl, R₁₄=tert-butoxycarbonyl, 2.75 g, 11.88 mmol) in dichloromethane (150 mL) was added compound 3-d (5.00 g, 14.25 mmol, 1.20 eq.), 4-dimethylaminopyridine (DMAP) (3.63 g, 29.60 mmol, 2.50 eq.) and 1-ethyl-3-(3-dimethylaminopropyl)carbodiimide hydrochloride (EDCI) (2.73 g, 14.25 mmol, 1.20 eq.). The mixture was stirred at room temperature for 18 hours. The reaction mixture was washed with aqueous 1 M potassium hydrogen sulfate, aqueous saturated sodium bicarbonate and brine. The organic layer was dried over magnesium sulfate and concentrated in vacuo. The residue was purified by column chromatography to give the title compound as a white solid (4.71 g, 75%).

¹H-NMR (CDCl₃): δ 7.29-7.39 (m, 7H), 6.24-6.30 (m, 1H), 5.29-5.37 (m, 1H), 5.13-5.18 (m, 2H), 4.73 (d, J=12.8 Hz, 1H), 4.23-4.55 (m, 3H), 4.10-4.21 (m, 1H), 2.99-3.16 (m, 1H), 2.46-2.62 (m, 1H), 1.48-1.79 (m, 4H), 1.43 (s, 9H), 0.95 (s, 9H).

### Step 5: [1-((S)-2-Amino-3,3-dimethyl-butyryl)-piperidin-4-yl]-furan-2-ylmethyl-carbamic acid benzyl ester hydrochloride (3-f)

Compound 3-e (4.71 g, 8.93 mmol) was dissolved in ethyl acetate (30 mL) and saturated with gaseous hydrochloric acid, and the reaction mixture stirred until all of the starting material was consumed. The mixture was concentrated to give amine hydrochloride salt as a white crystalline solid which was used in next step without further purification (4.10 g, 99%).

¹H-NMR (D₂O): δ 7.10-7.21 (m, 5H), 6.90-7.09 (m, 2H), 5.92-6.17 (m, 1H), 5.05-5.31 (m, 1H), 4.85-4.93 (m, 2H), 4.10-4.34 (m, 3H), 3.72-3.90 (m, 2H), 2.86-3.20 (m, 1H), 2.35-2.55 (m, 1H), 1.39-1.53 (m, 4H), 0.91 (s, 9H).

### General procedure IV

### Synthesis of [1-((S)-2-Amino-3,3-dimethyl-butyryl)-piperidin-4-yl]-(5-methoxy-4-oxo-4H-pyran-2-ylmethyl)-carbamic acid 2,2,2-trichloro-ethyl ester hydrochloride (Scheme 4)

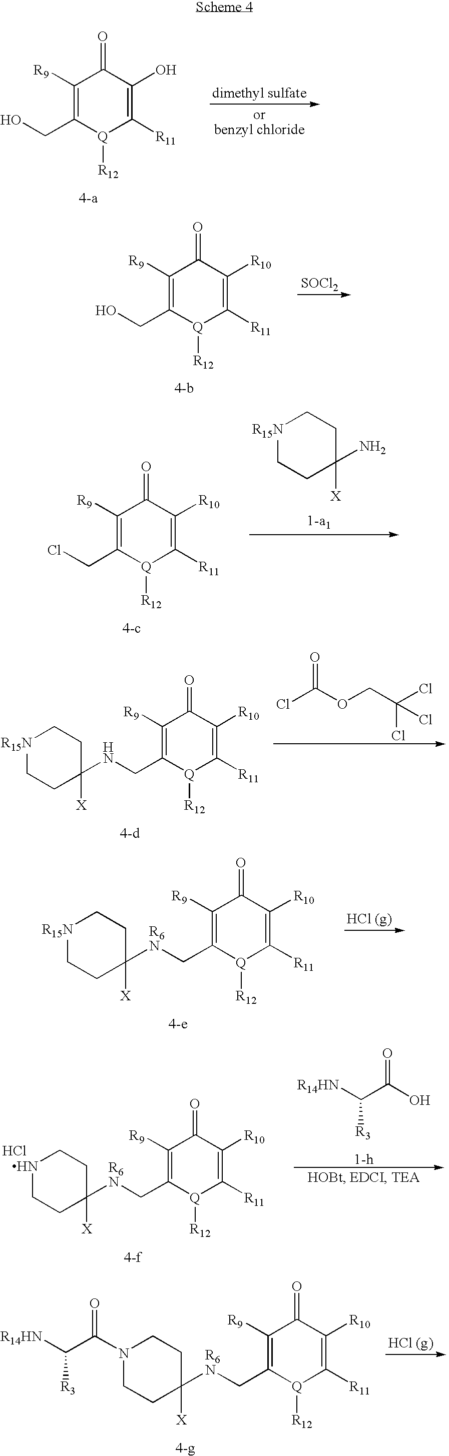

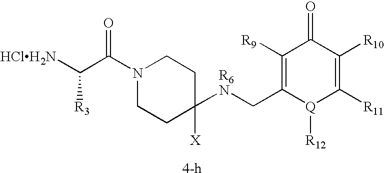

### Step 1-1: 2-Hydroxymethyl-5-methoxy-pyran-4-one (4-h, R₁₀=OMe)

A solution of compound 4-a (R₉═R₁₁═H, Q=O, 30.00 g, 211.10 mmol) in 10% aqueous potassium hydroxide (132 mL) was cooled to 0° C. Dimethyl sulfate (22.10 ml, 1.11 eq.) was slowly added and stirred at room temperature for 30 minutes. The reaction mixture was cooled to 0° C. and further stirred for 50 minutes. Yellow precipitate was removed by filtration and filtrate was concentrated in vacuo to give the title compound as a pale yellow (21.29 g, 65%).

¹H-NMR (DMSO-d₆): δ 8.08 (s, 1H), 6.29 (s, 1H), 5.72 (bs, 1H), 4.28 (s, 2H), 3.65 (s, 3H).

### Step 1-2: 5-Benzyloxy-2-hydroxymethyl-pyran-4-one (4-h, R₁₀=benzyloxy)

To a solution of compound 4-a (R₉═R₁₁═H, 30.00 g, 211.10 mmol) in methanol (211 mL) was added aqueous sodium hydroxide (9.30 g, 232.50 mmol, 1.10 eq.) solution (21 mL). Benzyl chloride (27.00 ml, 234.63 mmol, 1.11 eq.) was added to reaction mixture and refluxed for 17 hours. After methanol was concentrated in vacuo, residual solid was washed with H₂O (85 mL) and methanol (43 mL) to give the title compound as a white solid (38.38 g, 78%).

¹H-NMR (DMSO-d₆): δ 8.17 (s, 1H), 7.33-7.44 (m, 5H), 6.33 (s, 1H), 5.68 (bs, 1H), 4.95 (s, 2H), 4.30 (m. 2H).

### Step 2: 2-Chloromethyl-5-methoxy-pyran-4-one (4-c, R₁₀=OMe)

A solution of compound 4-h (3.50 g, 22.42 mmol) in thionyl chloride (12.80 ml, 175.48 mmol, 7.81 eq.) was stirred at room temperature for 1 hour. Hexane and ethyl acetate was added to the reaction mixture, the precipitate was washed with hexane and diethyl ether to give the title compound as a yellow solid (3.50 g, 89%).

¹H-NMR (DMSO-d₆): δ 8.20 (s, 1H), 6.54 (s, 1H), 4.67 (s, 2H), 3.66 (s, 3H).

### Step 3: 4-[(5-Methoxy-4-oxo-4H-pyran-2-ylmethyl)-amino]-piperidine-1-carboxylic acid tert-butyl ester (4-d)

To a solution of compound 1-a₁ (R₁₅=tert-butoxycarbonyl, X═H, 2.00 g, 9.99 mmol) in acetonitrile (80 mL) was added compound 4-c (1.75 g, 10.02 mmol, 1.00 eq.), N,N-diisopropylethylamine (3.50 ml, 20.09 mmol, 2.01 eq.) and refluxed for 17 hours. The reaction mixture was diluted with ethyl acetate and washed with aqueous saturated sodium bicarbonate. The organic layer was dried over magnesium sulfate and concentrated in vacuo. The residue was purified by column chromatography to give the title compound as a white solid (2.34 g, 69%).

¹H-NMR (CDCl₃): δ 7.54 (s, 1H), 6.43 (s, 1H), 3.95-4.02 (m, 2H), 3.77 (s, 3H), 3.69 (s, 2H), 2.80 (t, J=11.8 Hz, 2H), 2.60-2.67 (m, 1H), 1.82 (d, J=10.8 Hz, 2H), 1.45 (s, 9H), 1.23-1.32 (m, 2H).

### Step 4: 4-[(5-Methoxy-4-oxo-4H-pyran-2-ylmethyl)-(2,2,2-trichloro-ethoxycarbonyl)-amino]-piperidine-1-carboxylic acid tert-butyl ester (4-e, R₆=2,2,2-trichloroethoxycarbonyl)

To a solution of compound 4-d (2.18 g, 6.44 mmol) in acetonitrile (40 mL) was added pyridine (1.20 ml, 14.84 mmol, 2.30 eq) and 2,2,2-trichloroethyl chloroformate (1.10 ml, 7.99 mmol, 1.24 eq.). The reaction mixture was stirred at room temperature for 1 hour. After concentrated in vacuo, the reaction mixture was diluted with ethyl acetate, washed with 0.5 M aqueous citric acid and 0.6 M aqueous sodium bicarbonate. The organic layer was dried over magnesium sulfate and solvent was evaporated. The residue was purified by column chromatography to give the title compound as a orange solid (2.33 g, 70%).

¹H-NMR (CDCl₃): δ 7.52 (s, 1H), 6.35 (s, 1H), 4.78 (d, J=24.0 Hz, 2H), 4.22-4.30 (m, 4H), 3.77 (s, 3H), 2.74-2.77 (m, 2H), 1.76-1.79 (m, 2H), 1.51-1.61 (m, 3H), 1.45 (s, 9H).

### Step 5: (5-Methoxy-4-oxo-4H-pyran-2-ylmethyl)-piperidin-4-yl-carbamic acid 2,2,2-trichloro-ethyl ester hydrochloride (4-f)

Compound 4-e (2.23 g, 4.34 mmol) was dissolved in ethyl acetate (40 mL) and saturated with gaseous hydrochloric acid, and the reaction mixture stirred until all of the starting material was consumed. The mixture was concentrated to give amine hydrochloride salt as a white crystalline solid which was used in next step without further purification (1.95 g, 100%).

¹H-NMR (CD₃OD): δ 8.12 (s, 1H), 6.50 (s, 1H), 4.85-4.87 (m, 2H), 4.54 (s, 2H), 4.24-4.36 (m, 1H), 3.80 (s, 3H), 3.53 (d, J=11.6 Hz, 2H), 3.15 (t, J=12.6 Hz, 2H), 2.04-2.25 (m, 4H).

### Step 6: [1-((S)-2-tert-Butoxycarbonylamino-3,3-dimethyl-butyryl)-piperidin-4-yl]-(5-methoxy-4-oxo-4H-pyran-2-ylmethyl)-carbamic acid 2,2,2-trichloro-ethyl ester (4-g)

To a solution of compound I-h (R₃=tertbutyl, R₁₄=tert-butoxycarbonyl, 0.70 g, 3.03 mmol) in DMF (20 m) was added 1-hydroxybenzotriazole hydrate (HOBt) (0.49 g, 3.63 mmol, 1.20 eq.) and 1-ethyl-3-(3-dimethylaminopropyl)carbodiimide hydrochloride (EDCI) (0.70 g, 3.65 mmol, 1.20 eq.). After stirred for 80 min, compound 4-f (1.64 g, 3.64 mmol, 1.20 eq.) and triethylamine (1.06 ml, 7.61 mmol, 2.50 eq.) was added to the reaction mixture and stirred at room temperature for 16 hours. The reaction mixture was diluted with H₂O and extracted with ethyl acetate. The organic layer was washed with H₂O and aqueous saturated sodium bicarbonate and dried over magnesium sulfate. Solvent was evaporated to dryness and the residue was purified by column chromatography to give the title compound as a pale yellow solid (1.16 g, 61%).

¹H-NMR (CDCl₃): δ 7.51 (d, J=3.20 Hz, 1H), 6.35 (d, J=4.40 Hz, 1H), 5.28 (d, J=9.20 Hz, 1H), 4.74-4.84 (m, 3H), 4.48-4.54 (m, 1H), 4.23-4.30 (m, 3H), 3.76 (s, 3H), 3.11-3.17 (m, 2H), 2.53-2.65 (m, 1H), 1.77-1.85 (m, 2H), 1.50-1.68 (m, 3H), 1.43 (s, 9H), 0.97-1.01 (m, 9H).

### Step 7: [14 (S)-2-Amino-3,3-dimethyl-butyryl)-piperidin-4-yl]-(5-methoxy-4-oxo-4 H-pyran-2-ylmethyl)-carbamic acid 2,2,2-trichloro-ethyl ester hydrochloride (4-h)

Compound 4-g (1.08 g, 1.72 mmol) was dissolved in ethyl acetate (20 mL) and saturated with gaseous hydrochloric acid, and the reaction mixture stirred until all of the starting material was consumed. The mixture was concentrated to give amine hydrochloride salt as a white crystalline solid which was used in next step without further purification (0.97 g, 100%).

¹H-NMR (CD₃OD): δ 7.94 (s, 1H), 6.34 (s, 1H), 4.59 (d, J=12.4 Hz, 1H), 4.37-4.38 (m,2H), 4.09-4.19 (m, 2H), 3.66 (s, 3H), 3.12-3.17 (m, 2H), 2.65-2.71 (m, 1H), 1.70-1.80 (m, 6H), 0.98-1.02 (m, 10H).

### General Procedure V

### Synthesis of (R)-2-Cyclopentylmethyl-N¹-{(S)-2,2-dimethyl-1-[4-(4-methyl-benzylamino)-piperidine-1-carbonyl]-propyl}-N⁴-hydroxy-succinamide (Scheme 5)

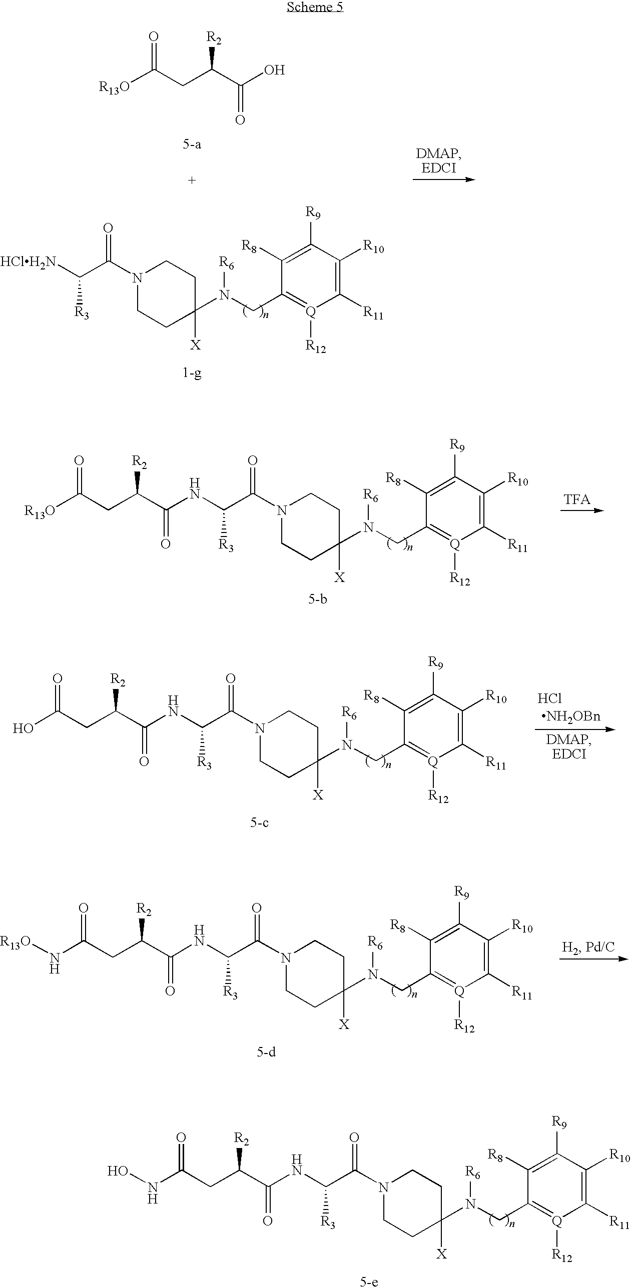

### Step 1: (R)-N-((S)-1-{4-[Benzyloxycarbonyl-(4-methyl-benzyl)-amino]-piperidine-1-carbonyl}-2,2-dim ethyl-propyl)-3-cyclopentylmethyl-succinic acid tert-butyl ester (5-b)

To a solution of compound 5-a (R₂=cyclopentylmethyl, R₁₃=tert-butyl, 1.00 g, 3.90 mmol) in dichloromethane (100 mL) was added 1-g (R₃=tert-butyl, R₆=benzyloxycarbonyl, R₈═R₉═R₁₁═R₁₂═X═H, R₁₀=Me, Q=C, n=1, 2.28 g, 4.68 mmol, 1.20 eq.), 4-dimethylaminopyridine (DMAP) (1.05 g, 9.75 mmol, 2.50 eq.) and 1-ethyl-3-(3-dimethylaminopropyl)carbodiimide hydrochloride (EDCI) (0.90 g, 4.68 mmol, 1.20 eq.). The mixture was stirred at room temperature for 18 hours. The reaction mixture was washed with aqueous 1 M potassium hydrogen sulfate, aqueous saturated sodium bicarbonate and brine. The organic layer was dried over magnesium sulfate and concentrated in vacuo. The residue was purified by column chromatography to give the title compound as a white (2.34 g, 87%).

¹H-NMR (CDCl₃): δ 7.16-7.43 (m, 5H), 6.95-7.12 (m, 4H), 6.16-6.30 (m, 1H), 5.10-5.20 (m, 2H), 4.91-5.05 (m, 1H), 4.72-4.91 (m, 2H), 4.63-4.75 (m, 1H), 4.05-4.35 (m, 4H), 3.51-3.75 (m, 2H), 2.70-2.91 (m, 1H), 2.40-2.64 (m, 2H), 2.32 (s, 3H), 1.60 (s, 9H), 1.32-1.80 (m, 12H), 0.99 (s, 9H).

### Step 2: (R)-N-((S)-1-{4-[Benzyloxycarbonyl-(4-methyl benzyl)-amino]-piperidine-1-carbonyl}-2,2-dim ethyl-propyl)-3-cyclopentylmethyl-succinic acid (5-c)

A solution of compound 5-b (2.00 g, 2.90 mmol) in dichloromethane (50 mL) was cooled to 0° C. Trifluoroacetic acid (10 mL) was slowly added and stirred at room temperature for 1 hour. The reaction mixture was evaporated under reduced pressure and treated with 1 N sodium hydroxide. After aqueous layer was washed with dichloromethane, adjusted to pH 2 with concentrated hydrochloric acid and extracted with dichloromethane. The organic layer was dried over magnesium sulfate and concentrated in vacuo to give the title compound as a white solid (1.65 g, 90%).

¹H-NMR (CDCl₃): δ 7.17-7.42 (m, 5H), 6%-7.10 (m, 4H), 6.10-6.27 (m, 1H), 5.12-5.22 (m, 2H), 4.91-5.05 (m, 1H), 4.71-4.90 (m, 2H), 4.59-4.70 (m, 1H), 4.04-4.35 (m, 4H), 3.50-3.73 (m, 2H), 2.71-2.92 (m, 1H), 2.40-2.63 (m, 2H), 2.30 (s, 3H), 1.31-1.80 (m, 12H), 1.01 (s, 9H).

### Step 3: {1-[(S)-2-((R)-3-Benzyloxycarbamoyl-2-cyclopentylmethyl-propionylamino)-3,3-dimethyl-butyryl]-piperidin-4-yl}-(4-methyl-benzyl)-carbamic acid benzyl ester (5-d)

To a solution of compound 5-c (1.50 g, 2.37 mmol) in dichloromethane (100 mL) was added O-benzylhydroxylamine hydrochloride (0.45 g, 2.84 mmol, 1.20 eq.), 4-dimethylaminopyridine (DMAP) (0.72 g, 5.92 mmol, 2.50 eq.) and 1-ethyl-3-(3-dimethylaminopropyl)carbodiimide hydrochloride (EDCI) (0.54 g, 2.84 mmol, 1.20 eq.). The mixture was stirred at room temperature for 18 hours. The reaction mixture was washed with 1 N hydrochloric acid, aqueous saturated sodium bicarbonate and brine. The organic layer was dried over magnesium sulfate and concentrated in vacuo. The residue was purified by column chromatography to give the title compound as a white solid (1.49 g, 85%).

¹H-NMR (CDCl₃): δ 7.16-7.50 (m, 10H), 6.92-7.12 (m, 4H), 6.15-6.32 (m, 1H), 5.13-5.20 (m, 2H), 4.94-5.08 (m, 1H), 4.72-4.91 (m, 4H), 4.61-4.71 (m, 1H), 4.05-4.35 (m, 4H), 3.51-3.75 (m, 2H), 2.70-2.91 (m, 1H), 2.42-2.62 (m, 2H), 2.33 (s, 3H), 1.31-1.84 (m, 12H), 1.00 (m, 9H).

### Step 4: (R)-2-Cyclopentylmethyl-N¹-{(S)-2,2-dimethyl-1-[4-(4-methyl-benzylamino)-piperidine-1-carbonyl]-propyl}-N⁴-hydroxy-succinamide (5-e)

To a solution of compound 5-d (1.26 g, 1.71 mmol) in ethanol (100 mL) was added 10 wt. % palladium on charcoal (0.18 mg). A balloon of hydrogen was placed over the reaction mixture and it was stirred for 2 hours. The charcoal was removed by filtration and the filtrate was concentrated to give a crude product. The crude product was purified by column chromatography to give the title compound as a white solid (0.60 g, 68%).

¹H-NMR (CDCl₃): δ 6.93-7.20 (m, 4H), 6.10-6.22 (m, 1H), 4.96-5.06 (m, 1H), 4.72-4.93 (m, 2H), 4.61-4.71 (m, 1H), 4.05-4.35 (m, 4H), 3.71-3.85 (s, 2H), 2.70-2.91 (m, 1H), 2.42-2.62 (m, 2H), 2.33 (s, 3H), 1.31-1.84 (m, 12H), 1.00 (m, 9H).

### General Procedure VI

### Synthesis of (R)-2-Cyclopentylmethyl-N-{(S)-2,2-dimethyl-1-[4-(4-methyl-benzylamino)-piperidine-1-carbonyl]-propyl}-3-(formyl-hydroxy-amino)-propionamide (Scheme 6)

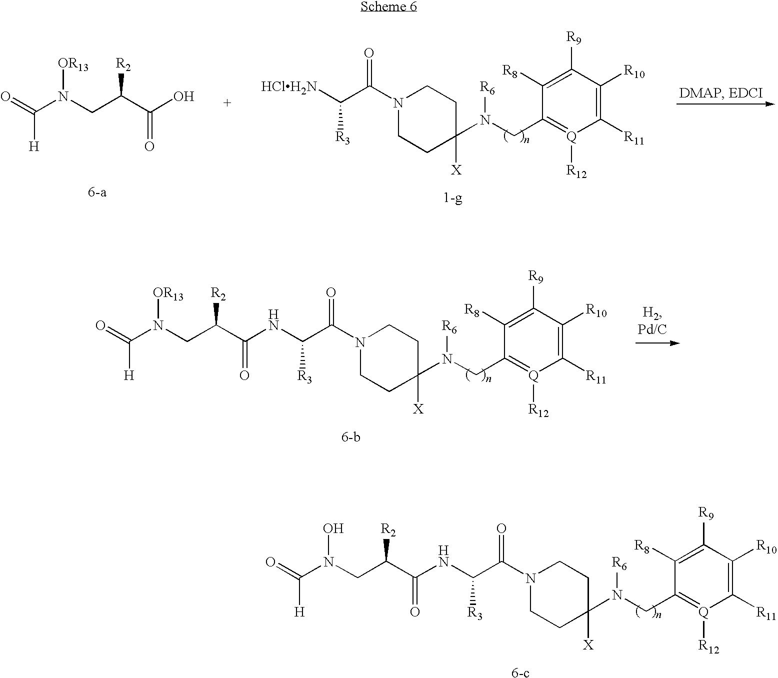

### Step 1: (1-{(S)-2-[(R)-3-(Benzyloxy-formyl-amino)-2-cyclopentylmethyl-propionylamino]-3,3-dimethyl-butyryl}-piperidin-4-yl)-(4-methyl-benzyl)-carbamic acid benzyl ester (6-h)

To a solution of compound 6-a (R₂=cyclopentylmethyl, R₁₃=benzyl, 0.70 g, 2.29 mmol) in dichloromethane (60 mL) was added 1-g (R₃=tert-butyl, R₆=benzyloxycarbonyl, R₈═R₉═R₁₁═R₁₂═X═H, R₁₀=Me, Q=C, n=1, 1.34 g, 2.75 mmol, 1.20 eq.), 4-dimethylaminopyridine (DMAP) (0.70 g, 5.73 mmol, 2.50 eq.) and 1-ethyl-3-(3-dimethylaminopropyl)carbodiimide hydrochloride (EDCI) (0.53 g, 2.75 mmol, 1.20 eq.). The mixture was stirred at room temperature for 18 hours. The reaction mixture was washed with aqueous 1 M potassium hydrogen sulfate, aqueous saturated sodium bicarbonate and brine. The organic layer was dried over magnesium sulfate and concentrated in vacuo. The residue was purified by column chromatography to give the title compound as a white (1.39 g, 82%).

¹H-NMR (CDCl₃): δ 8.11 (s, 0.6H), 7.85 (s, 0.4H), 7.13-7.48 (m, 10H), 6%-7.10 (m, 4H), 6.19-6.35 (m, 1H), 5.10-5.25 (m, 2H), 4.91-5.05 (m, 1H), 4.71-4.90 (m, 2H), 4.60-4.70 (m, 1H), 4.05-4.50 (m, 4H), 3.50-3.71 (m, 2H), 2.90-3.10 (m, 1H), 2.40-2.64 (m, 2H), 2.32 (s, 3H), 1.32-1.80 (m, 12H), 0.81-1.09 (m, 11H).

### Step 2: (R)-2-Cyclopentylmethyl-N-{(S)-2,2-dimethyl-1-[4-(4-methyl-benzyl)-piperidine-1-carbonyl]-propyl}-3-(formyl-hydr oxy-amino)-propionamide (6-c, R₆═H)

To a solution of compound 6-h (1.26 g, 1.71 mmol) in ethanol (100 mL) was added 10 wt. % palladium on charcoal (0.18 g). A balloon of hydrogen was placed over the reaction mixture and it was stirred for 2 hours. The charcoal was removed by filtration and the filtrate was concentrated to give a crude product. The crude product was purified by column chromatography to give the title compound as a pale yellow solid (0.49 g, 56%).

¹H-NMR (CDCl₃): δ 8.39 (s, 0.4H), 7.80 (s, 0.6H), 7.18-7.22 (m, 2H), 7.11-7.17 (m, 2H), 4.87-4.97 (m, 1H), 4.20-4.54 (m, 1H), 3.79 (s, 2H), 3.73-4.14 (m, 1H), 3.42-3.58 (m, 1H), 2.93-3.17 (m, 1H), 2.63-2.90 (m, 3H), 2.34 (s, 3H), 1.19-2.06 (m, 14H), 0.87-1.13 (m, 11H).

### General procedure VII

### Synthesis of (R)-N-{(S)-1-[4-Amino-4-(4-fluoro-benzyl)-piperidine-1-carbonyl]-2,2-dimethyl-propyl}-2-cyclopentylmethyl-3-(formyl-hydroxy-amino)-propionamide (Scheme 7)

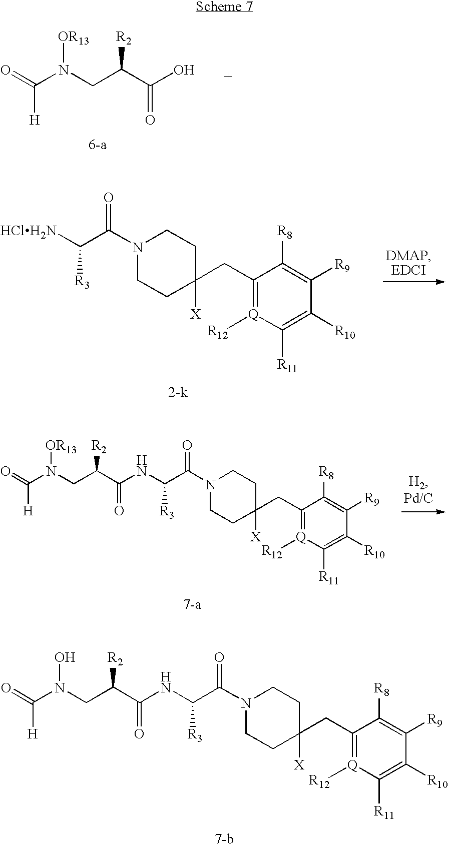

### Step 1: {1-{(S)-2-[(R)-3-(Benzyloxy)-formyl-amino)-2-cyclopentylmethyl-propionylamino]-3,3-dimethyl-butyryl}-4-(4-fluoro-benzyl)-piperidin-4-yl}-carbamic acid benzyl ester (7-a)

To a solution of compound 6-a (R₂=cyclopentylmethyl, R₁₃=benzyl, 0.70 g, 2.29 mmol) in dichloromethane (60 mL) was added 2-k (R₃=tert-butyl, R₈═R₉═R₁₁═R₁₂═H, R₁₀═F, Q=C, X=NHCbz, 1.35 g, 2.75 mmol, 1.20 eq.), 4-dimethylaminopyridine (DMAP) (0.70 g, 5.73 mmol, 2.50 eq.) and 1-ethyl-3-(3-dimethylaminopropyl)carbodiimide hydrochloride (EDCI) (0.53 g, 2.75 mmol, 1.20 eq.). The mixture was stirred at room temperature for 18 hours. The reaction mixture was washed with aqueous 1 M potassium hydrogen sulfate, aqueous saturated sodium bicarbonate and brine. The organic layer was dried over magnesium sulfate and concentrated in vacuo. The residue was purified by column chromatography to give the title compound as a (1.33 g, 78%).

¹H-NMR (CDCl₃): δ 8.11 (s, 0.7H), 7.82 (s, 0.3H), 7.01-7.49 (m, 12H), 6.88-6.99 (m, 2H), 5.09-5.25 (m, 1H), 4.25-4.35 (m, 2H), 3.50-3.71 (m, 2H), 3.23-3.34 (m, 2H), 2.90-3.10 (m, 2H), 2.65 (s, 2H), 2.13-2.25 (m, 2H), 1.30-1.80 (m, 14H), 0.80-1.10 (m, 11H).

### Step 2: (R)-N-{(S)-1-[4-Amino-4-(4-fluoro-benzyl)-piperidine-1-carbonyl]-2,2-dimethyl-propyl}-2-cyclopentylmethyl-3-(formyl-hydroxy-amino)-propionamide (7-h, X═NH₂)

To a solution of compound 7-a (1.20 g, 1.62 mmol) in ethanol (100 mL) was added 10 wt. % palladium on charcoal (0.17 g). A balloon of hydrogen was placed over the reaction mixture and it was stirred for 2 hours. The charcoal was removed by filtration and the filtrate was concentrated to give a crude product. The crude product was purified by column chromatography to give the title compound as a pale yellow solid (0.44 g, 52%).

¹H-NMR (CDCl₃): δ 8.45 (s, 0.3H), 7.80 (s, 0.7H), 7.40-7.50 (m, 2H), 6.91-7.10 (m, 2H), 4.95-5.10 (m, 1H), 3.75-3.97 (m, 2H), 2.77-2.95 (m, 2H), 2.70 (s, 2H), 2.45-2.75 (m, 2H), 1.35-2.10 (m, 14H), 0.85-1.10 (m, 11H).

### General Procedure

### Synthesis of (R)-2-Cyclopentylmethyl-3-(formyl-hydroxy-amino)-N-((S)-1-{4-[(furan-2-ylmethyl)-amino]-piperidine-1-carbonyl}-2,2-dimethyl-propyl)-propionamide (Scheme 8)

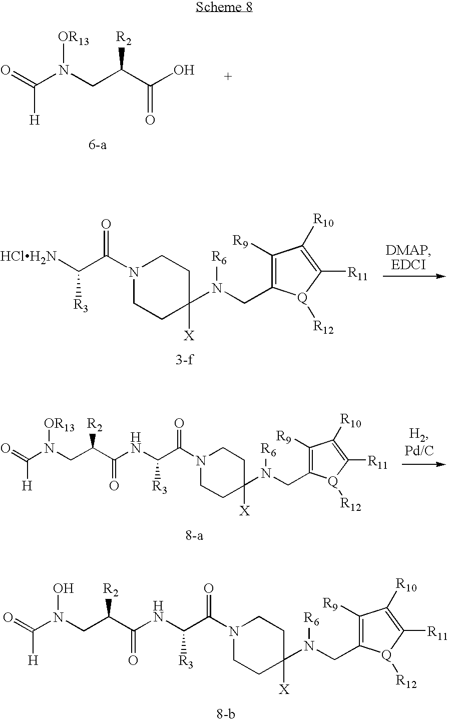

### Step 1: (1-{(S)-2-[(R)-3-(Benzyloxy-formyl-amino)-2-cyclopentylmethyl-propionylamino]-3,3-dimethyl-butyryl}-piperidin-4-yl)-furan-2-ylmethyl-carbamic acid benzyl ester (8-a)

To a solution of compound 6-a (R₂=cyclopentylmethyl, R₁₃=benzyl, 1.26 g, 4.13 mmol) in dichloromethane (60 mL) was added 3-f (R₃=tert-butyl, R₆=benzyloxycarbonyl, R₉═R₁₀═R₁₁═X═H, Q=O, 2.30 g, 4.96 mmol, 1.20 eq.), 4-dimethylaminopyridine (DMAP) (1.26 g, 10.33 mmol, 2.50 eq.) and 1-ethyl-3-(3-dimethylaminopropyl)carbodiimide hydrochloride (EDCI) (0.95 g, 4.96 mmol, 1.20 eq.). The mixture was stirred at room temperature for 18 hours. The reaction mixture was washed with aqueous 1 M potassium hydrogen sulfate, aqueous saturated sodium bicarbonate and brine. The organic layer was dried over magnesium sulfate and concentrated in vacuo. The residue was purified by column chromatography to give the title compound as a (1.75 g, 59%).

¹H-NMR (CDCl₃): δ 8.12 (s, 0.61), 7.86 (s, 0.4H), 7.25-7.48 (m, 12H), 6.22-6.30 (m, 1H), 5.11-5.19 (m, 2H), 4.66-5.05 (m, 4H), 4.08-4.48 (m, 4H), 3.64-3.82 (m, 2H), 2.99-3.10 (m, 2H), 2.40-2.69 (m, 2H), 1.20-1.90 (m, 12H), 0.80-1.10 (m, 11H)

### Step 2: (R)-2-Cyclopentylmethyl-3-(formyl-hydroxy-amino)-N-((S)-2-{4-[(furan-2-ylmethyl)-amino]-piperidine-1-carbonyl}-2,2-dimethyl-propyl)-propionamide (8-h, R₆═H)

To a solution of compound 8-a (1.75 g, 2.45 mmol) in ethanol (100 mL) was added 10 wt. % palladium on charcoal (0.24 g). A balloon of hydrogen was placed over the reaction mixture and it was stirred for 2 hours. The charcoal was removed by filtration and the filtrate was concentrated to give a crude product. The crude product was purified by column chromatography to give the title compound as a pale yellow solid (0.55 g, 46%).

¹H-NMR (CDCl₃): δ 8.39 (s, 0.3H), 7.81 (s. 0.7H), 7.31-7.38 (m, 1H), 6.29-6.33 (m, 1H), 6.14-6.19 (m, 1H), 4.85-4.97 (m, 1H), 4.20-4.55 (m, 1H), 3.97-4.13 (m, 1H), 3.79-3.84 (m, 2H), 3.41-3.57 (m, 1H), 3.05-3.17 (m, 1H), 2.65-2.90 (m, 3H), 1.16-1.98 (m, 14H), 0.88-1.13 (m, 11H).

### General Procedure IX

### Synthesis of (R)-2-Cyclopentylmethyl-3-(formyl-hydroxy-amino)-N-((S)-1-{4-[(5-methoxy-4-oxo-4H-pyran-2-ylmethyl)-amino]-piperidine-1-carbonyl}-2,2-dimethyl-propyl)-propionamide (Scheme 9)

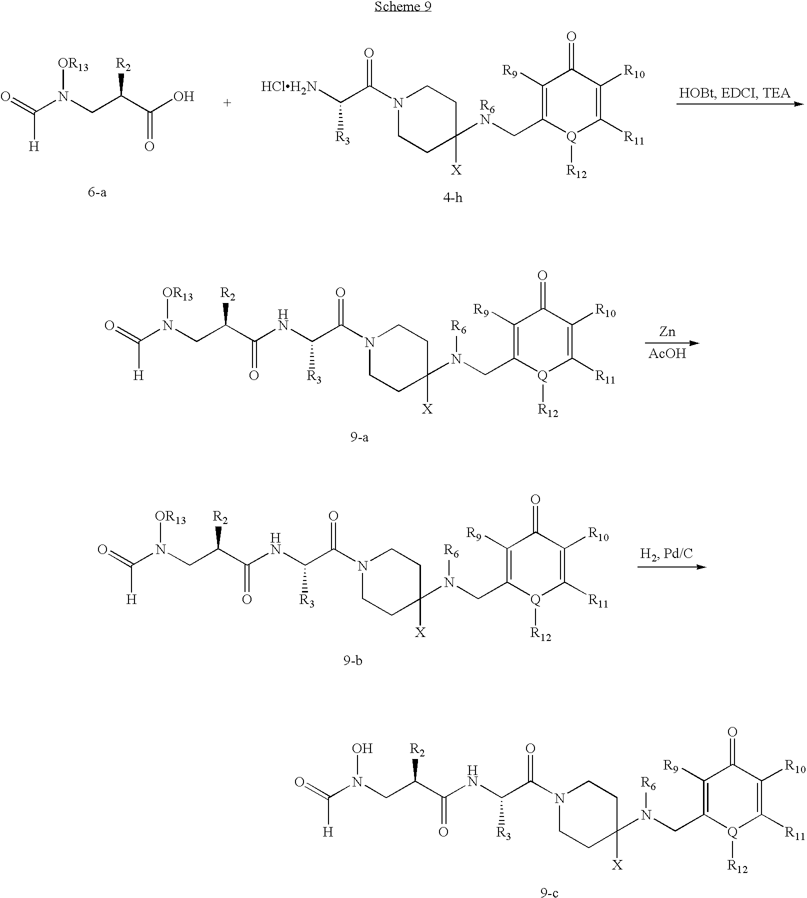

### Step 1: (1-{(S)-2-[(R)-3-(Benzyloxy-formyl-amino)-2-cyclopentylmethyl-propionylamino]-3,3-dimethyl-butyryl}-piperidin-4-yl)-(5-methoxy-4-oxo-4H-pyran-2-ylmethyl)-carbamic acid 2,2,2-trichloro-ethyl ester (9-a)

To a solution of compound 6-a (R₂=cyclopentylmethyl, R₁₃=benzyl, 0.40 g, 1.31 mmol) in DMF (10 m) was added 1-hydroxybenzotriazole hydrate (HOBt) (0.21 g, 1.55 mmol, 1.20 eq.) and 1-ethyl-3-(3-dimethylaminopropyl)carbodiimide hydrochloride (EDCI) (0.30 g, 1.56 mmol, 1.20 eq.). After stirred for 60 minutes, compound 4-h (R₃=tert-butyl, R₆=2,2,2-trichloroethoxycarbonyl, R₉═R₁₁═X═H, R₁₀=OMe, Q=0, 0.89 g, 1.58 mmol, 1.20 eq.) and triethylamine (0.46 ml, 3.30 mmol, 2.50 eq.) was added to the reaction mixture and stirred at room temperature for 15 hours. The reaction mixture was diluted with H₂O and extracted with ethyl acetate. The organic layer was washed with H₂O and aqueous saturated sodium bicarbonate and dried over magnesium sulfate. Solvent was evaporated to dryness and the residue was purified by column chromatography to give the title compound as a white (0.63 g, 59%).

¹H-NMR (CDCl₃): δ 8.12 (s, 0.6H), 7.84 (s, 0.4H), 7.51 (d, J=4.40 Hz, 1H), 7.37-7.41 (m, 5H), 6.34 (d, J=3.20 Hz, 1H), 6.21-6.28 (m, 1H), 4.74-4.84 (m, 5H), 4.21-4.36 (m, 4H), 3.66-3.77 (m, 4H), 3.05-3.16 (m, 1H), 1.45-1.88 (m, 16H), 0.89-1.05 (m, 11H).

### Step 2: (R)-3-(Benzyloxy-formyl-amino)-2-Cyclopentylmethyl-N-((S)-1-{4-[(5-methoxy-4-oxo-4H-pyran-2-ylmethyl)-amino]-piperidine-1-carbonyl}-2,2-dimethyl-propyl)-propionamide (9-b, R₆═H)

To a solution of compound 9-a (0.61 g, 0.69 mmol) in acetic acid (9 mL) was added zinc (0.65 g, 9.88 mmol, 14.00 eq.). The reaction mixture was stirred at room temperature for 17 hours. After insoluble material was removed by filtration, filtrate was extracted with ethyl acetate and washed with aqueous saturated sodium carbonate. The organic layer was dried over magnesium sulfate and concentrated in vacuo. The residue was purified by column chromatography to give the title compound as a pale yellow solid (0.39 g, 79%).

¹H-NMR (CDCl₃): δ 8.14 (s, 0.81), 7.87 (s, 0.4H), 7.54 (d, J=3.20 Hz, 1H), 7.37 (m, 5H), 6.42 (d, J=11.2 Hz, 1H), 6.28-6.34 (m, 1H), 4.79-5.00 (m, 3H), 4.10-4.22 (m, 1H), 3.67-3.77 (m, 5H), 2.97-3.11 (m, 1H), 2.69-2.80 (m, 2H), 1.83-1.95 (m, 2H), 1.12-1.74 (m, 14H), 0.89-1.03 (m, 11H).

### Step 3: (R)-2-Cyclopentylmethyl-3-(formyl-hydroxy-amino)-N-((S)-1-{4-[(5-methoxy-4-oxo-4H-pyran-2-ylmethyl)-amino]-piperidine-1-carbonyl}-2,2-dimethyl-propyl)-propionamide (9-c)

To a solution of compound 9-h (0.29 g, 0.45 mmol) in methanol (10 mL) was added 10 wt. % palladium on charcoal (0.13 g). A balloon of hydrogen was placed over the reaction mixture and it was stirred for 2 hours. The charcoal was removed by filtration and the filtrate was concentrated to give a crude product. The crude product was purified by column chromatography to give the title compound as a pale yellow solid (0.19 g, 77%).

¹H-NMR (CD₃OD): δ 8.27 (s, 0.3H), 7.83 (s, 0.7H), 4.95-5.00 (m, 1H), 4.36-4.56 (m, 1H), 3.99-4.29 (m, 2H), 3.69-3.86 (m, 3H), 3.41-3.47 (m, 3H), 3.32-3.34 (m, 2H), 3.15-3.22 (m, 1H), 3.03-3.09 (m, 1H), 2.66-2.90 (m, 4H), 1.27-2.07 (m, 11H), 0.99-1.20 (m, 11H).

### EXAMPLE 1

### (R)-2-Cyclopentylmethyl-N¹-{(S)-2,2-dimethyl-1-[4-(4-methyl-benzylamino)-piperidine-1-carbonyl]-propyl}-N⁴-hydroxy-succinamide

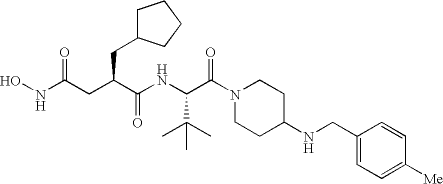

The title compound was prepared from (R)-2-cyclopentylmethyl-succinic acid 4-tert-butyl ester 5-a (R₂=cyclopentylmethyl, R₁₃=tert-butyl) and [1-((S)-2-amino-3,3-dimethyl-butyryl)-piperidin-4-yl]-(4-methyl-benzyl)-carbamic acid benzyl ester hydrochloride 1-g (prepared from General procedure I. R₃=tert-butyl, R₆=benzyloxycarbonyl, R₈═R₉═R₁₁═R₁₂═X═H, R₁₀=Me, Q=C, n=1) according to General procedure V.

¹H-NMR (CDCl₃): δ 6.93-7.20 (m, 4H), 6.10-6.22 (m, 1H), 4.96-5.06 (m, 1H), 4.72-4.93 (m, 2H), 4.61-4.71 (m, 1H), 4.05-4.35 (m, 4H), 3.71-3.85 (s, 2H), 2.70-2.91 (m, 1H), 2.42-2.62 (m, 2H), 2.33 (s, 3H), 1.31-1.84 (m, 12H), 1.00 (m, 9H).

### EXAMPLE 2

### (R)-2-Cyclopentylmethyl-N-{(S)-2,2-dimethyl-1-[4-(4-methyl-benzylamino)-piperidine-1-carbonyl]-propyl}-3-(formyl-hydroxy-amino)-propionamide

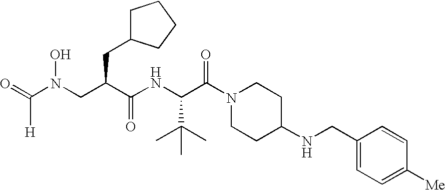

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(4-methyl-benzyl)-carbamic acid benzyl ester hydrochloride 1-g (prepared from General procedure I. R₃=tert-butyl, R₆=benzyloxycarbonyl, R₈═R₉═R₁₁═R₁₂═X═H, R₁₀=Me, Q=C, n=1) according to General procedure VI.

¹H-NMR (CDCl₃): δ 8.39 (s, 0.4H), 7.80 (s, 0.6H), 7.18-7.22 (m, 2H), 7.11-7.17 (m, 2H), 4.87-4.97 (m, 1H), 4.20-4.54 (m, 1H), 3.79 (s, 2H), 3.73-4.14 (m, 1H), 3.42-3.58 (m, 1H), 2.93-3.17 (m, 1H), 2.63-2.90 (m, 3H), 2.34 (s, 3H), 1.19-2.06 (m, 14H), 0.87-1.13 (m, 11H).

### EXAMPLE 3

### (R)-N-{(S)-1-[4-(4-Cyano-benzylamino)-piperidine-1-carbonyl]-2,2-dimethyl-propyl}-2-cyclopentylmethyl-3-(formyl-hydroxy-amino)-propionamide

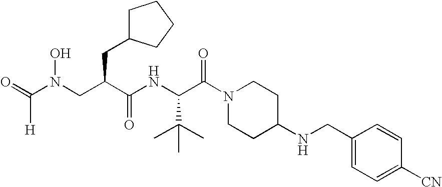

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(4-cyano-benzyl)-carbamic acid benzyl ester hydrochloride 1-g (prepared from General procedure I. R₃=tert-butyl, R₆=benzyloxycarbonyl, R₈═R₉═R₁₁═R₁₂═X═H, R₁₀═CN, Q=C, n=1) according to General procedure VI

¹H-NMR (CDCl₃): δ 8.39 (s, 0.3H), 7.81 (s, 0.7H), 7.57-7.65 (m, 2H), 7.40-7.49 (m, 2H), 4.85-4.97 (m, 1H), 4.18-4.56 (m, 1H), 3.96-4.16 (m, 1H), 3.89 (s, 2H), 3.40-3.57 (m, 1H), 2.95-3.18 (m, 1H), 2.65-2.92 (m, 3H), 1.17-2.06 (m, 14H), 0.83-1.13 (m, 11H).

### EXAMPLE 4

### (R)-2-Cyclopentylmethyl-N-{(S)-1-[4-(4-fluoro-benzylamino)-piperidine-1-carbonyl]-2,2-dimethyl-propyl}-3-(formyl-hydroxy-amino)-propionamide

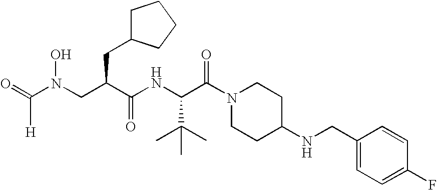

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(4-fluoro-benzyl)-carbamic acid benzyl ester hydrochloride 1-g (prepared from General procedure I. R₃=tert-butyl, R₆=benzyloxycarbonyl, R₈═R₉═R₁₁═R₁₂═X═H, R₁₀═F, Q=C, n=1) according to General procedure VI.

¹H-NMR (CDCl₃): δ 8.39 (s, 0.3H), 7.81 (s, 0.7H), 7.21-7.31 (m, 2H), 6.95-7.05 (m, 2H), 4.85-4.98 (m, 1H), 4.18-4.55 (m, 1H), 3.92-4.13 (m, 1H), 3.78 (s, 2H), 3.40-3.55 (m, 1H), 2.94-3.18 (m, 1H), 2.65-2.91 (m, 3H), 1.16-2.05 (m, 14H), 0.87-1.13 (m, 11H).

### EXAMPLE 5

### (R)-2-Cyclopentylmethyl-3-(formyl-hydroxy-amino)-N-{(S)-1-[4-(4-methoxy-benzyl amino)-piperidine-1-carbonyl]-2,2-dimethyl-propyl}-propionamide

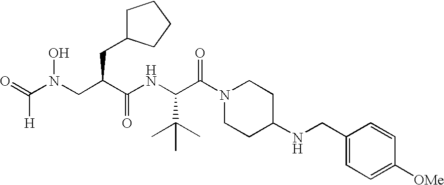

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(4-methoxy-benzyl)-carbamic acid benzyl ester hydrochloride 1-g (prepared from General procedure I. R₃=tert-butyl, R₆=benzyloxycarbonyl, R₈═R₉═R₁₁═R₁₂═X═H, R₁₀=OMe, Q=C, n=1) according to General procedure VI.

¹H-NMR (CDCl₃): δ 8.39 (s, 0.3H), 7.80 (s, 0.7H), 7.20-7.26 (m, 2H), 6.83-6.90 (m, 2H), 4.87-4.97 (m, 1H), 4.21-4.57 (m, 1H), 3.96-4.14 (m, 1H), 3.80 (s, 3H), 3.77 (s, 2H), 3.42-3.56 (m, 1H), 2.91-3.17 (m, 1H), 2.65-2.91 (m, 3H), 1.18-2.08 (m, 14), 0.88-1.14 (m, 11H).

### EXAMPLE 6

### (R)-2-Cyclopentylmethyl-3-(formyl-hydroxy-amino)-N-{(S)-1-[4-(4-hydroxy-benzyl amino)-piperidine-1-carbonyl]-2,2-dimethyl-formyl}-propionamide

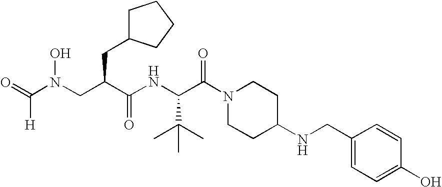

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(4-hydroxy-benzyl)-carbamic acid benzyl ester hydrochloride 1-g (prepared from General procedure I. R₃=tert-butyl, R₆=benzyloxycarbonyl, R₈═R₉═R₁₁═R₁₂═X═H, R₁₀═OH, Q=C, n=1) according to General procedure VI.

¹H-NMR (CD₃OD): δ 8.28 (s, 0.3H), 7.79 (s, 0.7H), 7.19 (d, J=8.0 Hz, 2H), 6.76 (d, J=8.4 Hz, 2H), 4.87-4.95 (m, 1H), 4.38-4.51 (m, 1H), 4.18-4.21 (m, 1H), 3.75-3.83 (m, 2H), 3.38-3.63 (m, 1H), 2.81-3.21 (m, 3H), 2.60-2.80 (m, 1H), 1.20-2.08 (m, 14H), 0.88-1.16 (m, 11H).

### EXAMPLE 7

### 4-[(1-{(S)-2-[(R)-2-Cyclopentylmethyl-3-(formyl-hydroxy-amino)-propionylamino]-3,3-dimethyl-butyryl}-piperidin-4-ylamino)-methyl]-benzoic acid methyl ester

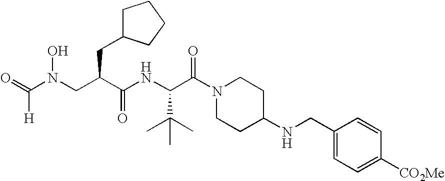

The title compound was prepared from (R)-3-(benzyloxy-formyl-amino)-2-cyclopentylmethyl-propionic acid 6-a (R₂=cyclopentylmethyl, R₁₃=benzyl) and 4-({[1-(S)-2-amino-3,3-dimethyl-butyryl]-piperidin-4-yl}-benzyloxycarbonylamino)-methyl)-benzoic acid methyl ester hydrochloride 1-g (prepared from General procedure I. R₃=tert-butyl, R₆=benzyloxycarbonyl, R₈═R₉═R₁₁═R₁₂═X═H, R₁₀═C(═O)OMe, Q=C, n=1) according to General procedure VI.

¹H-NMR (CDCl₃): δ 8.40 (s, 0.3H), 7.97-8.13 (m, 2H), 7.81 (s, 0.7H), 7.38-7.43 (m, 2H), 4.87-4.97 (m, 1H), 4.18-4.53 (m, 1H), 4.04-4.16 (m, 1H), 3.91 (s, 3H), 3.89 (s, 2H), 3.40-3.85 (m, 1H), 2.98-3.30 (m, 1H), 2.65-2.92 (m, 3H), 1.18-2.07 (m, 14H), 0.80-1.12 (m, 11H).

### EXAMPLE 8

### (R)-N-{(S)-1-[4-(4-Acetylamino-benzylamino)-piperidine-1-carbonyl]-2,2-dimethyl-propyl}-2-cyclopentylmethyl-3-(formyl-hydroxy-amino)-propionamide

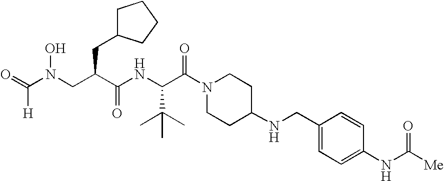

The title compound was prepared from (R)-3-(benzyloxy-formyl-amino)-2-cyclopentylmethyl-propionic acid 6-a (R₂=cyclopentylmethyl, R₁₃=benzyl) and 4-(acetylamino-benzyl)-[1-(S)-2-amino-3,3-dimethyl-butyryl]-piperidin-4-yl]-carbamic acid benzyl ester hydrochloride 1-g (prepared from General procedure I. R₃=tert-butyl, R₆=benzyloxycarbonyl, R₈═R₉═R₁₁═R₁₂═X═H, R₁₀═NHC(═O)Me, Q=C, n=1) according to General procedure VI.

¹H-NMR (CDCl₃): δ 8.39 (s, 0.3H), 7.81 (s, 0.7H), 7.53-7.68 (m, 2H), 6.99-7.10 (m, 2H), 4.88-4.99 (m, 1H), 4.17-4.57 (m, 1H), 3.92-4.17 (m, 1H), 3.77 (s, 2H), 3.45-3.65 (m, 1H), 2.95-3.15 (m, 1H), 2.64-2.93 (m, 3H), 2.41 (s, 3H), 1.13-2.09 (m, 14H), 0.89-1.13 (m, 11H).

### EXAMPLE 9

### (R)-2-Cyclopentylmethyl-N-((S)-2,2-dimethyl-1-{4-[(pyridine-2-ylmethyl)-amino]-piperidine-1-carbonyl}-propyl)-3-(formyl-hydroxy-amino)-propionamide

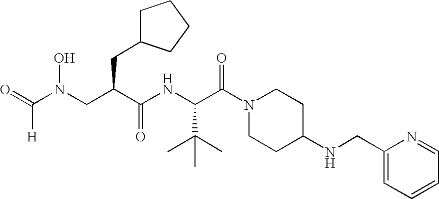

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-pyridin-2-ylmethyl-carbamic acid benzyl ester hydrochloride 1-g (prepared from General procedure I. R₃=tert-butyl, R₆=benzyloxycarbonyl, R₈═R₉═R₁₀═R₁₁═X═H, Q=N, n=1) according to General procedure VI.

¹H-NMR (CDCl₃): δ 8.52-8.58 (m, 1H), 8.38 (s, 0.3H), 7.81 (s, 0.7H), 7.61-7.68 (m, 1H), 7.27-7.33 (m, 1H), 7.15-7.21 (m, 1H), 4.88-4.97 (m, 1H), 4.23-4.56 (m, 1H), 3.98-4.14 (m, 1H), 3.94 (s, 2H), 3.40-3.57 (m, 1H), 2.94-3.17 (m, 1H), 2.65-2.89 (m, 3H), 1.20-2.04 (m, 14H), 0.88-1.13 (m, 11H).

### EXAMPLE 10

### (R)-2-Cyclopentylmethyl-N-{(S)-1-[4-(2,4-difluoro-benzylamino)-piperidine-1-carbonyl]-2,2-dimethyl-propyl}-3-(formyl-hydroxy-amino)-propionamide

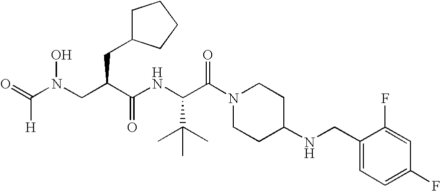

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(2,4-difluoro-benzyl)-carbamic acid benzyl ester hydrochloride 1-g (prepared from General procedure I. R₃=tert-butyl, R₆=benzyloxycarbonyl, R₉═R₁₁═R₁₂═X═H, R₈═R₁₀═F, Q=C, n=1) according to General procedure VI.

¹H-NMR (CDCl₃): δ 8.40 (s, 0.3H), 7.82 (s, 0.7H), 7.28-7.36 (m, 1H), 6.76-6.89 (m, 2H), 4.84-4.95 (m, 1H), 4.15-4.53 (m, 1H), 3.93-4.11 (m, 1H), 3.75 (s, 2H), 3.44-3.56 (m, 1H), 2.93-3.11 (m, 1H), 2.62-2.85 (m, 3H), 1.12-2.03 (m, 14H), 0.88-1.10 (m, 11H).

### EXAMPLE 11

### (R)-2-Cyclopentylmethyl-N-{(S)-1-[4-(2,4-dimethyl-benzylamino)-piperidine-1-carbonyl]-2,2-dimethyl-propyl}-3-(formyl-hydroxy-amino)-propionamide

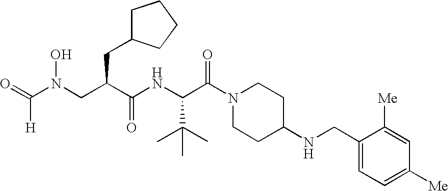

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(2,4-dimethyl-benzyl)-carbamic acid benzyl ester hydrochloride 1-g (prepared from General procedure I. R₃=tert-butyl, R₆=benzyloxycarbonyl, R₉═R₁₁═R₁₂═X═H, R₈═R₁₀=Me, Q=C, n=1) according to General procedure VI.

¹H-NMR (CDCl₃): δ 8.41 (s, 0.4H), 7.85 (s, 0.6H), 7.11-7.18 (m, 1H), 6.94-7.01 (m, 2H), 4.84-4.95 (m, 1H), 4.21-4.55 (m, 1H), 3.78 (s, 2H), 3.73-4.17 (m, 1H), 3.43-3.55 (m, 1H), 2.92-3.15 (m, 1H), 2.65-2.90 (m, 3H), 2.32-2.30 (s, 3H), 2.30-2.28 (s, 3H), 1.18-2.03 (m, 14H), 0.97-1.12 (m, 11H).

### EXAMPLE 12

### (R)-2-Cyclopentylmethyl-N-{(S)-1-[4-(2,4-dimethoxy-benzylamino)-piperidine-1-carbonyl]-2,2-dimethyl-propyl}-3-(formyl-hydroxy-amino)-propionamide

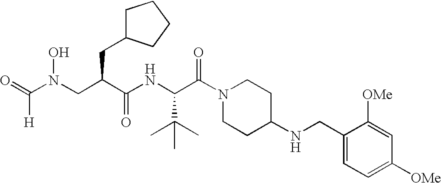

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(2,4-dimethoxy-benzyl)-carbamic acid benzyl ester hydrochloride 1-g (prepared from General procedure I. R₃=tert-butyl, R₆=benzyloxycarbonyl, R₉═R₁₁═R₁₂═X═H, R₈═R₁₀=OMe, Q=C, n=1) according to General procedure VI.

¹H-NMR (CDCl₃): δ 8.36 (d, J=3.2 Hz, 0.3H), 7.76 (d, J=64 Hz, 0.7H), 7.15-7.20 (m, 1H), 6.42-6.49 (m, 2H), 4.87-4.97 (m, 1H), 4.31-4.57 (m, 1H), 4.00-4.14 (m, 1H), 3.86-3.88 (m, 2H), 3.79 (s, 3H), 3.81 (s, 3H), 3.33-3.58 (m, 1H), 3.00-3.22 (m, 1H), 2.60-2.99 (m, 3H), 1.91-2.00 (m, 2H), 1.21-1.81 (m, 12H), 0.89-1.11 (m, 11H).

### EXAMPLE 13

### (R)-2-Cyclopentylmethyl-N-{(S)-1-[4-(3,4-dihydroxy-benzylamino)-piperidine-1-carbonyl]-2,2-dimethyl-propyl}-3-(formyl-hydroxy-amino)-propionamide

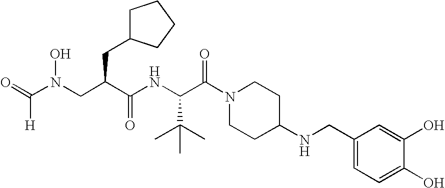

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(3,4-dihydroxy-benzyl)-carbamic acid benzyl ester hydrochloride 1-g (prepared from General procedure I. R₃=tert-butyl, R₆=benzyloxycarbonyl, R₈═R₁₁═R₁₂═X═H, R₉═R₁₀═OH, Q=C, n=1) according to General procedure VI.

¹H-NMR (CD₃OD): δ 8.25 (s, 0.3H), 7.91 (s, 0.7H), 6.78 (s, 1H), 6.73 (d, J=8.0 Hz, 1H), 6.67 (d, J=8.0 Hz, 1H), 4.93-4.96 (m, 1H), 4.36-4.58 (m, 1H), 4.17-4.30 (m, 1H), 3.67-3.74 (m, 2H), 3.37-3.61 (m, 1H), 2.61-3.21 (m, 4H), 1.18-2.09 (m, 14H), 0.94-1.15 (m, 11H).

### EXAMPLE 14

### (R)-2-Cyclopentylmethyl-N-{(S)-2,2-dimethyl-1-[4-(2,4,5-trifluoro-benzylamino)-piperidine-1-carbonyl]-propyl}-3-(formyl-hydroxy-amino)-propionamide

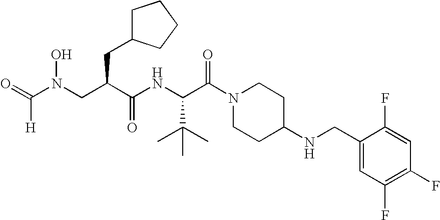

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(2,4,5-trifluoro-benzyl)-carbamic acid benzyl ester hydrochloride 1-g (prepared from General procedure I. R₃=tert-butyl, R₆=benzyloxycarbonyl, R₉═R₁₂═X═H, R₈═R₁₀═R₁₁═F, Q=C, n=1) according to General procedure VI.

¹H-NMR (CDCl₃): δ 8.40 (s, 0.3H), 7.82 (s, 0.7H), 7.22-7.26 (m, 1H), 6.70-6.72 (m, 1H), 4.91-4.96 (m, 1H), 4.18-4.54 (m, 1H), 3.96-4.15 (m, 1H), 3.81-3.82 (m, 2H), 3.43-3.48 (m, 1H), 2.68-3.35 (m, 4H), 1.84-2.03 (m, 2H), 1.18-1.82 (m, 12H), 0.93-1.13 (m, 11H).

### EXAMPLE 15

### (R)-2-Cyclopentylmethyl-N-[(S)-2,2-dimethyl-1-(4-phenylamino-piperidine-1-carbonyl)-propyl]-3-(formyl-hydroxy-amino)-propionamide

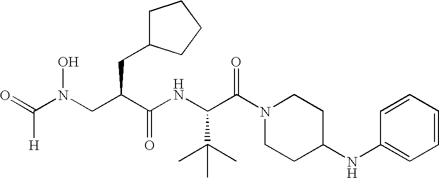

The title compound was prepared from (R)-3-(benzyloxy-formyl-amino)-2-cyclopentylmethyl-propionic acid 6-a (R₂=cyclopentylmethyl, R₁₃=benzyl) and (S)-2-amino-3,3-dimethyl-1-(4-phenylamino-piperidin-1-yl)-butan-1-one hydrochloride 1-g (prepared from General procedure I. R₃=tert-butyl, R₆═R₈═R₉═R₁₀═R₁₁═R₁₂═X═H, Q=C, n=0) according to General procedure VI.

¹H-NMR (CD₃OD): δ 8.26 (s, 0.3H), 7.82 (s, 0.7H), 7.07-7.12 (m, 2H), 6.59-6.68 (m, 3H), 4.96-5.00 (m, 1H), 4.10-4.50 (m, 2H), 3.52-3.61 (m, 1H), 3.02-3.13 (m, 1H), 2.82-2.93 (m, 1H), 1.98-2.13 (m, 2H), 1.87 (m, 1H), 1.19-1.74 (m, 12H), 0.99-1.14 (m, 11H).

### EXAMPLE 16

### (R)-N-{(S)-1-[4-(4-Cyano-phenylamino)-piperidine-1-carbonyl]-2,2-dimethyl-propyl}-2-cyclopentylmethyl-3-(formyl-hydroxy-amino)-propionamide

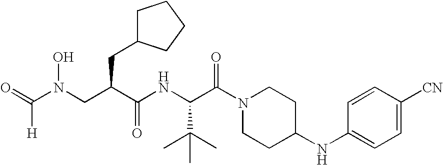

The title compound was prepared from (R)-3-(benzyloxy-formyl-amino)-2-cyclopentylmethyl-propionic acid 6-a (R₂=cyclopentylmethyl, R₁₃=benzyl) and 4-[1-((S)-2-amino-3,3-dimethyl-butyryl)-piperidin-4-ylamino]-benzonitrile hydrochloride 1-g (prepared from General procedure I. R₃=tert-butyl, R₆═R₈═R₉═R₁₁═R₁₂═X═H, R₁₀═CN, Q=C, n=0) according to General procedure VI.

¹H-NMR (CD₃OD): δ 8.27 (s, 0.3H), 7.83 (s, 0.7H), 7.39-7.42 (m, 2H), 6.68-6.71 (m, 2H), 4.97-5.01 (m, 1H), 4.09-4.53 (m, 2H), 3.57-3.72 (m, 1H), 3.04-3.15 (m, 1H), 2.85-2.91 (m, 1H), 1.99-2.13 (m, 2H), 1.87-1.91 (m, 1H), 1.24-1.73 (m, 12H), 1.00-1.09 (m, 11H).

### EXAMPLE 17

### (R)-2-Cyclopentylmethyl-N-{(S)-1-[4-(4-fluoro-phenylamino)-piperidine-1-carbonyl]-2,2-dimethyl-propyl}-3-(formyl-hydroxy-amino)-propionamide

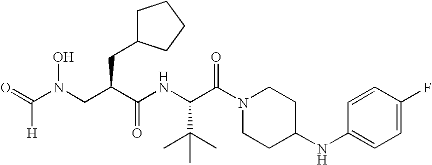

The title compound was prepared from (R)-3-(benzyloxy-formyl-amino)-2-cyclopentylmethyl-propionic acid 6-a (R₂=cyclopentylmethyl, R₁₃=benzyl) and (S)-2-amino-1-[4-(4-fluoro-phenylamino)-piperidin-1-yl]-3,3-dimethyl-butan-1-one hydrochloride 1-g (prepared from General procedure I. R₃=tert-butyl, R₆═R₈═R₉═R₁₁═R₁₂═X═H, R₁₀═CN, Q=C, n=0) according to General procedure VI.

¹H-NMR (CD₃OD): δ 8.24 (s, 0.3H), 7.80 (s, 0.7H), 6.81-6.86 (m, 2H), 6.62-6.66 (m, 2H), 4.94-4.97 (m, 1H), 4.08-4.48 (m, 2H), 3.71-3.79 (m, 1H), 3.38-3.59 (m, 2H), 3.02-3.06 (m, 1H), 2.80-2.86 (m, 1H), 1.95-2.09 (m, 2H), 1.85-1.86 (m, 1H), 1.18-1.72 (m, 12H), 0.97-1.12 (m, 11H).

### EXAMPLE 18

### (R)-2-Cyclopentylmethyl-N-[(S)-2,2-dimethyl-1-(4-p-tolylamino-piperidine-1-carbonyl)-propyl]-3-(formyl-hydroxy-amino)-propionamide

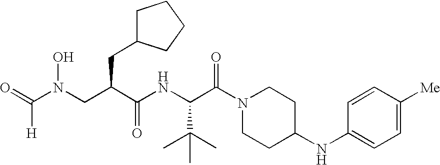

The title compound was prepared from (R)-3-(benzyloxy-formyl-amino)-2-cyclopentylmethyl-propionic acid 6-a (R₂=cyclopentylmethyl, R₁₃=benzyl) and (S)-2-amino-3,3-dimethyl-1-(4-p-tolylamino-piperidin-1-yl)-butan-1-one hydrochloride 1-g (prepared from General procedure I. R₃=tertbutyl, R₆═R₈═R₉═R₁₁═R₁₂═X═H, R₁₀=Me, Q=C, n=0) according to General procedure VI.

¹H-NMR (CD₃OD): δ 8.22 (s, 0.3H), 7.77 (s, 0.7H), 6.88-6.90 (m, 2H), 6.56 (t, J=7.4 Hz, 2H), 4.92-4.95 (m, 1H), 4.06-4.46 (m, 2H), 3.69-3.75 (m, 1H), 3.43-3.57 (m, 1H), 3.31-3.41 (m, 1H), 2.99-3.02 (m, 1H), 2.76-2.87 (m, 1H), 2.15 (s, 3H), 1.93-2.08 (m, 2H), 1.77-1.82 (m, 1H), 1.15-1.70 (m, 12H), 0.94-1.10 (m, 11H).

### EXAMPLE 19

### (R)-2-Cyclopentylmethyl-3-(formyl-hydroxy-amino)-N-{(S)-1-[4-(4-methoxy-phenyl amino)-piperidine-1-carbonyl]-2,2-dimethyl-propyl}-propionamide

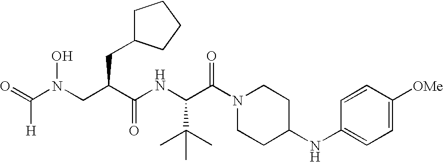

The title compound was prepared from (R)-3-(benzyloxy-formyl-amino)-2-cyclopentylmethyl-propionic acid 6-a (R₂=cyclopentylmethyl, R₁₃=benzyl) and (S)-2-amino-1-[4-(4-methoxy-phenylamino)-piperidin-1-yl]-3,3-dimethyl-butan-1-one hydrochloride 1-g (prepared from General procedure I. R₃=tert-butyl, R₆═R₈═R₉═R₁₁═R₁₂═X═H, R₁₀=Me, Q=C, n=0) according to General procedure VI.

¹H-NMR (CD₃OD): δ 8.26 (s, 0.3H), 7.81 (s, 0.7H), 6.66-6.77 (m, 4H), 4.95-4.98 (m, 1H), 4.11-4.50 (m, 2H), 3.72-3.81 (m, 1H), 3.71 (s, 3H), 3.39-3.49 (m, 2H), 3.00-3.08 (m, 1H), 2.79-2.90 (m, 1H), 1.96-2.10 (m, 2H), 1.16-1.91 (m, 12H), 0.98-1.10 (m, 11H).

### EXAMPLE 20

### (R)-2-Cyclopentylmethyl-3-(formyl-hydroxy-amino)-N-{(S)-1-[4-(4-hydroxy-phenyl amino)-piperidine-1-carbonyl]-2,2-dimethyl-formyl}-propionamide

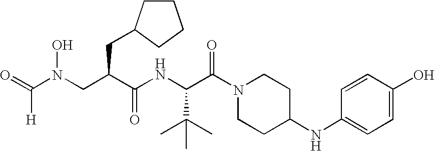

The title compound was prepared from (R)-3-(benzyloxy-formyl-amino)-2-cyclopentylmethyl-propionic acid 6-a (R₂=cyclopentylmethyl, R₁₃=benzyl) and (S)-2-amino-1-[4-(4-hydroxy-phenylamino)-piperidin-1-yl]-3,3-dimethyl-butan-1-one hydrochloride 1-g (prepared from General procedure I. R₃=tert-butyl, R₆═R₈═R₉═R₁₁═R₁₂═X═H, R₁₀═OH, Q=C, n=0) according to General procedure VI.

¹H-NMR (CD₃OD): δ 8.26 (s, 0.3H), 7.81 (s, 0.7H), 6.88-6.95 (m, 2H), 6.75-6.80 (m, 2H), 4.92-4.93 (m, 1H), 4.22-4.59 (m, 2H), 3.72-3.84 (m, 1H), 3.52-3.60 (m, 1H), 3.39-3.48 (m, 1H), 3.17-3.24 (m, 1H), 2.83-2.95 (m, 1H), 2.70-2.77 (m, 1H), 1.98-2.08 (m, 2H), 1.29-1.87 (m, 12H), 0.98-1.08 (m, 11H).

### EXAMPLE 21

### 4-(1-[(S)-2-[(R)-2-Cyclopentylmethyl-3-(formyl-hydroxy-amino)-propionylamino]-3,3-dimethyl-butyryl]-piperidin-4-ylamino)-benzoic acid methyl ester

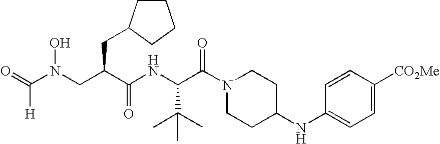

The title compound was prepared from (R)-3-(benzyloxy-formyl-amino)-2-cyclopentylmethyl-propionic acid 6-a (R₂=cyclopentylmethyl, R₁₃=benzyl) and 4-[1-((S)-2-amino-3,3-dimethyl-butyryl)-piperidin-4-ylamino]-benzoic acid dimethyl ester hydrochloride 1-g (prepared from General procedure I. R₃=tert-butyl, R₆═R₈═R₉═R₁₁═R₁₂═X═H, R₁₀═C(═O)OMe, Q=C, n=0) according to General procedure VI.

¹H-NMR (CD₃OD): δ 8.26 (s, 0.3H), 7.82 (s, 0.7H), 7.75-7.77 (m, 2H), 6.61-6.65 (m, 2H), 4.96-5.00 (m, 1H), 4.11-4.51 (m, 2H), 3.81 (s, 3H), 3.73-3.79 (m, 1H), 3.56-3.70 (m, 1H), 3.40-3.46 (m, 1H), 3.04-3.15 (m, 1H), 2.83-2.91 (m, 1H), 1.99-2.08 (m, 2H), 1.23-1.89 (m, 12H), 0.99-1.09 (m, 11H).

### EXAMPLE 22

### (R)-2-Cyclopentylmethyl-N-{(S)-2,2-dimethyl-1-[4-(methyl-phenyl-amino)-piperidin e-1-carbonyl]-propyl}-3-(formyl-hydroxy-amino)-propionamide

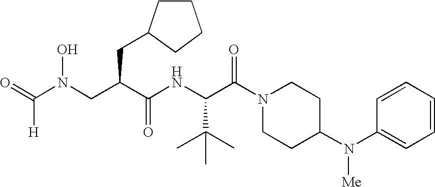

The title compound was prepared from (R)-3-(benzyloxy-formyl-amino)-2-cyclopentylmethyl-propionic acid 6-a (R₂=cyclopentylmethyl, R₁₃=benzyl) and (S)-2-amino-3,3-dimethyl-1-[4-(methyl-phenyl-amino)-piperidin-1-yl]-butan-1-one hydrochloride 1-g (prepared from General procedure I. R₃=tert-butyl, R₈═R₉═R₁₀═R₁₁═R₁₂═X═H, R₆=Me, Q=C, n=0) according to General procedure VI.

¹H-NMR (CD₃OD): δ 8.23 (s, 0.3H), 7.79 (s, 0.7H), 7.14-7.18 (m, 2H), 6.86 (t, J=7.6

Hz, 2H), 6.65-6.70 (m, 1H), 4.91-5.00 (m, 1H), 4.59-4.63 (m, 1H), 4.29-4.33 (m, 1H), 3.87-3.93 (m, 1H), 3.69-3.79 (m, 1H), 3.37-3.58 (m, 1H), 3.14-3.24 (m, 1H), 2.90-3.08 (m, 1H), 2.63-2.73 (m, 4H), 1.26-1.88 (m, 14H), 0.95-1.12 (m, 11H).

### EXAMPLE 23

### (R)-2-Cyclopentylmethyl-N-{(S)-1-[4-(2,4-difluoro-phenylamino)-piperidine-1-carbonyl]-2,2-dimethyl-propyl}-3-(formyl-hydroxy-amino)-propionamide

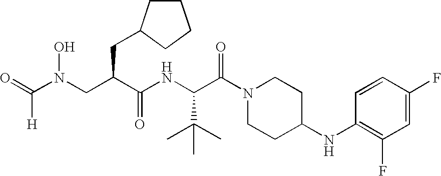

The title compound was prepared from (R)-3-(benzyloxy-formyl-amino)-2-cyclopentylmethyl-propionic acid 6-a (R₂=cyclopentylmethyl, R₁₃=benzyl) and (S)-2-amino-1-[4-(2,4-difluoro-phenylamino)-piperidin-1-yl]-3,3-dimethyl-butan-1-one hydrochloride 1-g (prepared from General procedure I. R₃=tert-butyl, R₆═R₉═R₁₁═R₁₂═X═H, R₈═R₁₀═F, Q=C, n=0) according to General procedure VI.

¹H-NMR (CD₃OD): δ 8.24 (s, 0.3H), 7.80 (s, 0.7H), 6.75-6.85 (m, 3H), 4.94-4.97 (m, 1H), 4.13-4.52 (m, 2H), 3.71-3.79 (m, 1H), 3.50-3.59 (m, 1H), 3.38-3.43 (m, 1H), 2.95-3.08 (m, 1H), 2.76-2.89 (m, 1H), 1.98-2.11 (m, 2H), 1.79-1.84 (m, 1H), 1.17-1.72 (m, 12H), 0.97-1.14 (m, 11H).

### EXAMPLE 24

### (R)-2-Cyclopentylmethyl-N-{(S)-1-[4-(2,4-dimethoxy-phenylamino)-piperidine-1-carbonyl]-2,2-dimethyl-propyl}-3-(formyl-hydroxy-amino)-propionamide

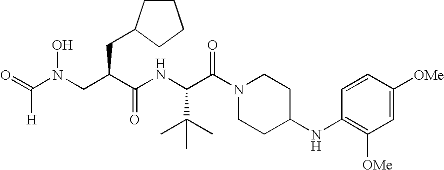

The title compound was prepared from (R)-3-(benzyloxy-formyl-amino)-2-cyclopentylmethyl-propionic acid 6-a (R₂=cyclopentylmethyl, R₁₃=benzyl) and (S)-2-amino-1-[4-(2,4-dimethoxy-phenylamino)-piperidin-1-yl]-3,3-dimethyl-butan-1-one hydrochloride 1-g (prepared from General procedure I. R₃=tert-butyl, R₆═R₉═R₁₁═R₁₂═X═H, R₈═R₁₀=OMe, Q=C, n=0) according to General procedure VI.

¹H-NMR (CD₃OD): δ 8.27 (s, 0.3H), 7.83 (s, 0.7H), 6.70-6.73 (m, 1H), 6.51-6.52 (m, 1H), 6.43 (dd, J=2.6, 8.6 Hz, 1H), 4.96-5.01 (m, 1H), 4.12-4.55 (m, 2H), 3.81-3.83 (m, 3H), 3.74 (s, 3H), 3.41-3.53 (m, 2H), 3.36 (s, 3H), 3.04-3.07 (m, 1H), 2.79-2.92 (m, 1H), 2.04-2.11 (m, 2H), 1.25-1.88 (m, 12H), 0.99-1.17 (m, 11H).

### EXAMPLE 25

### (R)-2-Cyclopentylmethyl-N-{(S)-1-[4-(3-fluoro-4-morpholin-4-yl-phenylamino)-piperidine-1-carbonyl]-2,2-dimethyl-propyl}-3-(formyl-hydroxy-amino)-propionamide

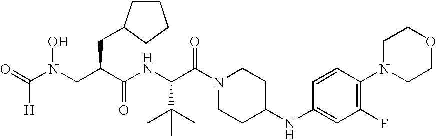

The title compound was prepared from (R)-3-(benzyloxy-formyl-amino)-2-cyclopentylmethyl-propionic acid 6-a (R₂=cyclopentylmethyl, R₁₃=benzyl) and (S)-2-amino-1-[4-(3-fluoro-4-morpholin-4-yl-phenylamino)-piperidin-1-yl]-3,3-dimethyl-butan-1-one hydrochloride 1-g (prepared from General procedure I. R₃=tert-butyl, R₆═R₈═R₁₁═R₁₂═X═H, R₉═F, R₁₀=morpholinyl, Q=C, n=0) according to General procedure VI.

¹H-NMR (CD₃OD): δ 8.25 (s, 0.3H), 7.81 (s, 0.7H), 6.85-6.90 (m, 1H), 6.40-6.47 (m, 2H), 4.94-4.99 (m, 1H), 4.08-4.48 (m, 2H), 3.73-3.81 (m, 5H), 3.38-3.52 (m, 2H), 3.02-3.13 (m, 1H), 2.82-2.94 (m, 5H), 1.96-2.10 (m, 2H), 1.16-1.86 (m, 12H), 0.98-1.10 (m, 11H).

### EXAMPLE 26

### (R)-N-{(S)-1-[4-Amino-4-(4-fluoro-benzyl)-piperidine-1-carbonyl]-2,2-dimethyl-propyl}-2-cyclopentylmethyl-3-(formyl-hydroxy-amino)-propionamide

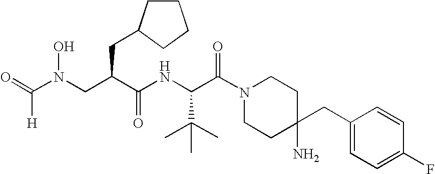

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-4-(4-fluoro-benzyl)-piperidin-4-yl]-carbamic acid benzyl ester hydrochloride 2-k (prepared from General procedure II. R₃=tert-butyl, R₈═R₉═R₁₁═R₁₂═H, R₁₀═F, Q=C, X═NHCbz) according to General procedure VII.

¹H-NMR (CDCl₃): δ 8.45 (s, 0.3H), 7.80 (s, 0.7H), 7.40-7.50 (m, 2H), 6.91-7.10 (m, 2H), 4.95-5.10 (m, 1H), 3.75-3.97 (m, 2H), 2.77-2.95 (m, 2H), 2.70 (s, 2H), 2.45-2.75 (m, 2H), 1.35-2.10 (m, 14H), 0.85-1.10 (m, 11H).

### EXAMPLE 27

### (R)-2-Cyclopentylmethyl-3-(formyl-hydroxy-amino)-N-((S)-1-{4-[(furan-2-ylmethyl)-amino]-piperidine-1-carbonyl}-2,2-dimethyl-propyl)-propionamide

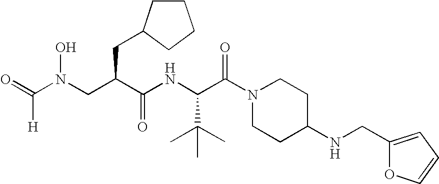

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-furan-2-ylmethyl-carbamic acid benzyl ester hydrochloride 3-f (prepared from General procedure III. R₃=tert-butyl, R₆=benzyloxycarbonyl, R₉═R₁₀═R₁₁═X═H, Q=0) according to General procedure VIII.

¹H-NMR (CDCl₃): δ 8.39 (s, 0.3H), 7.81 (s. 0.7H), 7.31-7.38 (m, 1H), 6.29-6.33 (m, 1H), 6.14-6.19 (m, 1H), 4.85-4.97 (m, 1H), 4.20-4.55 (m, 1H), 3.97-4.13 (m, 1H), 3.79-3.84 (m, 2H), 3.41-3.57 (m, 1H), 3.05-3.17 (m, 1H), 2.65-2.90 (m, 3H), 1.16-1.98 (m, 14H), 0.88-1.13 (m, 11H).

### EXAMPLE 28

### (R)-2-Cyclopentylmethyl-N-((S)-2,2-dimethyl-1-{4-[(1H-pyrrol-2-ylmethyl)-amino]-piperidine-1-carbonyl}-propyl)-3-(formyl-hydroxy-amino)-propionamide

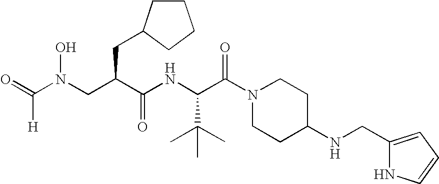

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(1H-pyrrol-2-ylmethyl)-carbamic acid benzyl ester hydrochloride 3-f (prepared from General procedure III. R₃=tert-butyl, R₆=benzyloxycarbonyl, R₉═R₁₀═R₁₁═R₁₂═X═H, Q=N) according to General procedure VIII.

¹H-NMR (CDCl₃): δ 8.39 (s, 0.3H), 7.82 (s. 0.7H), 6.71-6.75 (m, 1H), 6.11-6.16 (m, 1H), 5.99-6.02 (m, 1H), 4.85-4.93 (m, 1H), 4.22-4.54 (m, 1H), 3.57-4.12 (m, 1H), 3.75-3.85 (m, 2H), 3.41-3.560 (m, 1H), 3.03-3.16 (m, 1H), 2.66-2.93 (m, 3H), 1.15-1.96 (m, 14H), 0.87-1.12 (m, 11H).

### EXAMPLE 29

### (R)-2-Cyclopentylmethyl-3-(formyl-hydroxy-amino)-N-((S)-1-{4-[(5-methoxy-4-oxo-4H-pyran-2-ylmethyl)-amino]-piperidine-1-carbonyl}-2,2-dimethyl-propyl)-propionamide

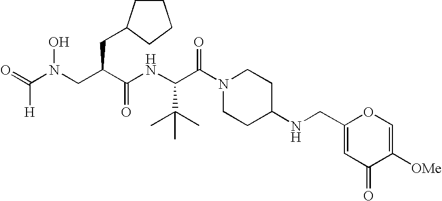

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(5-methoxy-4-oxo-4H-pyran-2-ylmethyl)-carbamic acid 2,2,2-trichloro-ethyl ester hydrochloride 4-h (prepared from General procedure W. R₃=tert-butyl, R₆=2,2,2-trichloroethoxycarbonyl, R₉═R₁₁═X═H, R₁₀=OMe, Q=0) according to General procedure IX.

¹H-NMR (CD₃OD): δ 8.27 (s, 0.3H), 7.83 (s, 0.7H), 4.95-5.00 (m, 1H), 4.36-4.56 (m, 1H), 3.99-4.29 (m, 2H), 3.69-3.86 (m, 3H), 3.41-3.47 (m, 3H), 3.32-3.34 (m, 2H), 3.15-3.22 (m, 1H), 3.03-3.09 (m, 1H), 2.66-2.90 (m, 4H), 1.27-2.07 (m, 11H), 0.99-1.20 (m, 11H).

### EXAMPLE 30

### (R)-2-Cyclopentylmethyl-3-(formyl-hydroxy-amino)-N-((S)-1-{4-[(5-hydroxy-4-oxo-4H-pyran-2-ylmethyl)-amino]-piperidin-1-carbonyl}-2,2-dimethyl-propyl)-propionamide

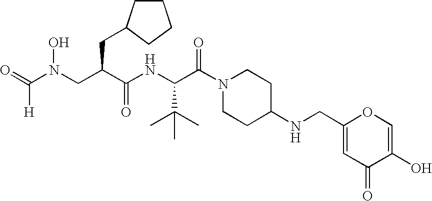

The title compound was prepared from (R)-3-(benzyloxy-formyl-amino)-2-cyclopentylmethyl-propionic acid 6-a (R₂=cyclopentylmethyl, R₁₃=benzyl) and [1-((S)-2-amino-3,3-dimethyl-butyryl)-piperidin-4-yl]-(5-benzyloxy-4-oxo-4H-pyran-2-ylmethyl)-carbamic acid 2,2,2-trichloro-ethyl ester hydrochloride 4-h (prepared from General procedure W. R₃=tert-butyl, R₆=2,2,2-trichloroethoxycarbonyl, R₉═R₁₁═X═H, R₁₀=OBn, Q=0) according to General procedure IX.

¹H-NMR (CD₃OD): δ 8.28 (s, 0.3H), 7.83 (s, 0.7H), 4.95-5.00 (m, 1H), 4.42-4.57 (m, 1H), 4.20-4.30 (m, 1H), 3.41-3.94 (m, 4H), 3.32-3.34 (m, 2H), 3.04-3.23 (m, 2H), 2.68-2.89 (m, 4H), 1.24-2.03 (m, 11H), 0.99-1.10 (m, 11H).

### Experimental Example

### 1. Test for Enzyme Activity

Activities of PDF enzyme for E. coli and S. aureus were determined using PDF/FDH coupled assay. In this coupled assay, the formate released by PDF from its substrate formyl-Methionine-Alanine-Serine (fMAS) is oxidized by coupling enzyme formate dehydrogenase (FDH), reducing one molecule of NAD⁺ to NADH, which is measured absorption at 340 nm.

IC₅₀ (nM) for some of the compounds of the Examples are reported in Table 1.

TABLE 1

| | Escherichia | Streptococcus | Streptococcus |
|---|---|---|---|
| compounds | coli | Pneumoniae | pyogenes |
| Example 1 | 150 | 65 | 70 |
| Example 2 | 132 | 77 | 86 |
| Example 6 | 102 | 42 | 84 |
| Example 10 | 122 | 55 | 83 |
| Example 13 | 133 | 46 | 88 |
| Example 20 | 102 | 63 | 61 |
| Example 22 | 113 | 25 | 42 |
| Example 27 | 117 | 42 | 63 |
| Example 29 | 155 | 43 | 54 |
| Example 30 | 160 | 50 | 62 |

### 2. Test for Antibacterial Activity

Minimum inhibitory concentrations (MICs) were determined using the microdilution method in 96-well format plates. Each of the compounds of Examples was dissolved in dimethyl sulfoxide to a concentration of 2 mg/mL and stored at 4° C. until used. They were diluted in Mueller-Hinton Broth (MHB) and used for MIC determination. The range of concentrations tested was 64-0.00625 g/mL final concentration using a two-fold dilution system. Plates were incubated at 37° C. and MIC were recorded after 24 hours of incubation for bacteria. MIC was defined as the lowest concentration of compound that does not produce visible growth after incubation.

Linezolid and vancomycin were used as standard antibiotics, respectively.

Results for some of the compounds of the Examples are reported in Table 2.

TABLE 2

| | MIC (μg/mL) | | | | |
|---|---|---|---|---|---|
| | Vancomycin- | Vancomycin- | | | |
| | resistant | resistant | Streptococcus | Streptococcus | |
| | Enterococcus | Enterococcus | Pneumoniae | Pneumoniae | Streptococcus |
| Compounds | faecalis | faecium | Pen NS | Pen S | pyogenes |
| Example 2 | 3.1 | 0.4 | 0.4 | 0.1 | 0.2 |
| Example 3 | 0.8 | 0.4 | 0.4 | 0.1 | 0.2 |
| Example 4 | 3.1 | 0.4 | 0.4 | 0.2 | 0.4 |
| Example 5 | 6.3 | 1.6 | 0.8 | 0.2 | 0.4 |
| Example 6 | >50 | 50 | 3.1 | 0.8 | 0.8 |
| Example 7 | 6.3 | 3.1 | 1.6 | 0.4 | 0.8 |
| Example 9 | 12.5 | 12.5 | 1.6 | 1.6 | 0.8 |
| Example 12 | 6.3 | 3.1 | 0.8 | 0.2 | 0.2 |
| Example 14 | 1.6 | 0.2 | 0.4 | 0.1 | 0.1 |
| Example 15 | 1.6 | 0.4 | 0.4 | 0.1 | 0.1 |
| Example 16 | 3.1 | 0.4 | 1.6 | 0.1 | 0.1 |
| Example 17 | 3.1 | 0.4 | 0.8 | 0.2 | 0.2 |
| Example 18 | 3.1 | 0.4 | 0.4 | 0.2 | 0.1 |
| Example 19 | 3.1 | 1.6 | 0.2 | 0.4 | 0.2 |
| Example 20 | 25 | 25 | 3.1 | 0.8 | 1.6 |
| Example 21 | 6.3 | 1.6 | 0.8 | 0.2 | 0.4 |
| Example 22 | 3.1 | 0.2 | 0.2 | 0.05 | 0.05 |
| Example 23 | 3.1 | 0.2 | 0.2 | 0.05 | 0.1 |
| Example 24 | 6.3 | 0.8 | 0.4 | 0.2 | 0.1 |
| Example 25 | 12.5 | 3.1 | 1.6 | 0.4 | 0.8 |
| Linezolid | 0.8 | 1.6 | 0.8 | 0.8 | 0.8 |
| Vancomycin | >50 | >50 | 0.4 | 0.2 | 0.4 |

### 3. Acute Toxicity

To demonstrate the usefulness of the compounds of this invention as medicaments we have performed acute toxicity study in mice.

The acute toxicity of the compounds of Example 5, 17 and 22 were tested using several groups of ICR mice each of 6 mice. 4,000 mg/kg dose of the medicament was each orally injected into each group of mice, and weight change and death were observed for 14 days after the injection.

Results of the compounds of the Examples are reported in Table 3.

TABLE 3

| Compounds | LD₅₀ (mg/kg) |
|---|---|
| Example 5 | >2,000 |
| Example 17 | >2,000 |
| Example 22 | >2,000 |

### INDUSTRIAL APPLICABILITY

The compounds of this invention, e.g. of formula (I) or a pharmaceutically acceptable salt thereof have low toxicity and are antibacterially active against gram-positive organisms, in particular also against those microorganisms which are resistant to various antibiotics. Thus, the compounds of this invention are useful as antibacterial agents for infection with resistant bacteria.

1. A compound of formula (I), all such racemic mixtures, optical isomers and diastereoisomers or a pharmaceutically acceptable salts thereof: 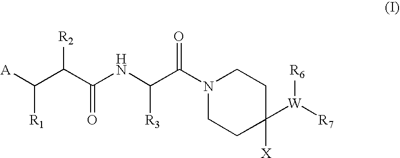
wherein, A is selected from the group of consisting of —C(═O)NHOH or —N(CHO)OH;
R₁ represents hydrogen, C₁₋₃ alkyl, C₄₋₆ cycloalkyl, halogen or hydroxy group;
R₂ represents hydrogen, straight or branched C₁₋₆ alkyl, straight or branched C₂₋₆ alkenyl, C₄₋₆ cycloalkyl, C₄₋₆ heterocycle including nitrogen or oxygen, or benzyl group;
R₃ represents hydrogen, straight or branched C₁₋₆ alkyl, straight or branched C₂₋₆ alkenyl, C₄₋₆ cycloalkyl, phenyl or benzyl group;
X represents hydrogen or NR₄R₅;
Each of R₄ and R₅ is independently hydrogen, straight or branched C₁₋₃ alkyl, tert-butoxycarbonyl, benzyloxycarbonyl group;
W represents carbon or nitrogen;
Each of R₆ and R₇ is independently hydrogen, straight or branched C₁₋₃ alkyl, tert-butoxylcarbonyl, benzyloxycarbonyl, 2,2,2-trichloroethoxycarbonyl or a group of formula (IIa), or (IIb), or (IIc): 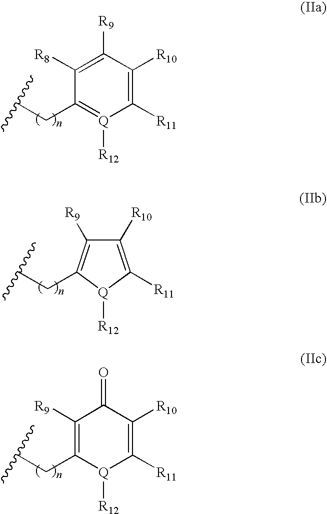
wherein, each of R₈, R₉, R₁₀, R₁₁ and R₁₂ is independently hydrogen, straight or branched C₁₋₃ alkyl, straight or branched C₁₋₃ alkylamine, C₃₋₆ cycloalkyl, C₄₋₆ heterocycle, C₁₋₃ alkoxyl, C₁₋₃ acyl, C₁₋₃ acyloxy, hydroxy, amido, halogen (fluoro, chloro, bromo and iodo), halogen-substituted C₁₋₃ alkyl, cyano, nitro or morpholinyl group;
Q represents carbon or nitrogen or oxygen;
n is 0 or 1 or 2.

2. The compound of formula (I) according to claim 1, wherein A is —C(═O)NHOH, R₁ is hydrogen, R₂ is iso butyl, n-butyl, n-pentyl, benzyl or cyclopentylmethyl, R₃ is tert-butyl, iso-propyl, phenyl or benzyl, W is carbon or nitrogen, X is hydrogen, amino, methylamino or dimethylamino, R₆ is hydrogen, methyl or ethyl, n is 0 or 1 or 2, Q is carbon or nitrogen or oxygen, each of R₈, R₉, R₁₀, R₁₁ and R₁₂ is independently hydrogen, methyl, fluoro, chloro, bromo, trifluoromethyl, methoxy, —C(═O)OMe, —NH(C═O)Me, cyano, hydroxy, nitro or morpholinyl; or a pharmaceutically acceptable salts thereof.

3. The compound of formula (I) according to claim 1, wherein A is N(CHO)OH, R₁ is hydrogen, R₂ is iso-butyl, n-butyl, n-pentyl, benzyl or cyclopentylmethyl, R₃ is tert-butyl, iso-propyl, phenyl or benzyl, W is carbon or nitrogen, X is hydrogen, amino, methylamino or dimethylamino, R₆ is hydrogen, methyl or ethyl, n is 0 or 1 or 2, Q is carbon or nitrogen or oxygen, each of R₈, R₉, R₁₀, R₁₁ and R₁₂ is independently hydrogen, methyl, fluoro, chloro, bromo, trifluoromethyl, methoxy, —C(═O)OMe, —NH(C═O)Me, cyano, hydroxy, nitro or morpholinyl; or a pharmaceutically acceptable salts thereof.

4. A process for preparing a compound of formula (I) or a pharmaceutically acceptable salt thereof, which comprises reacting a compound of formula (III) with hydroxylamine or an N- and/or O-protected hydroxylamine, and thereafter removing any N- or O-protecting groups: 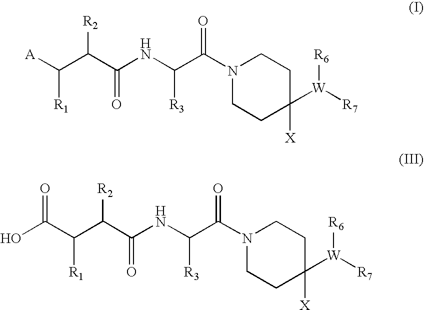
wherein, A R₁, R₂, R₃, R₆, R₇, W and X are the same as defined in claim 1.

5. The method for preparing a compound of formula (III) according to claim 4 which process comprises reacting a compound of formula (IV) with a compound of formula (Va) (or Vb, or Vc) or salt thereof: 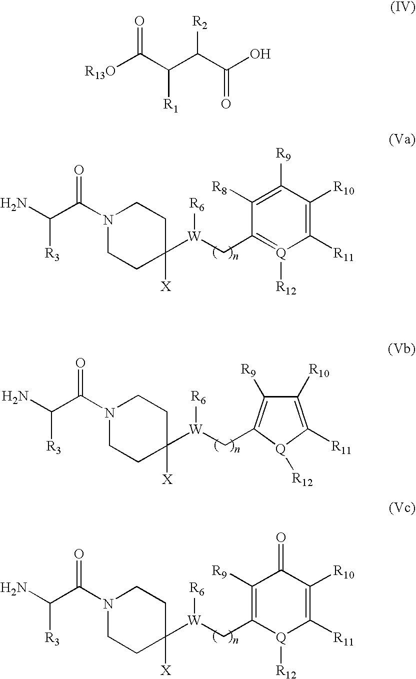
wherein, R₁, R₂, R₃, R₆, R₈, R₉, R₁₀, R₁₁, R₁₂, Q, W, X and n are the same as defined in claim 1 and R₁₃ is a protecting group, such as methyl, ethyl, tert-butyl and benzyl.

6. A process for preparing a compound of formula (I) or a pharmaceutically acceptable salt thereof, which comprises reacting a compound of formula (VI) with a compound of formula (Va) (or Vb, or Vc) or salt thereof, and thereafter removing any N- or O-protecting groups: 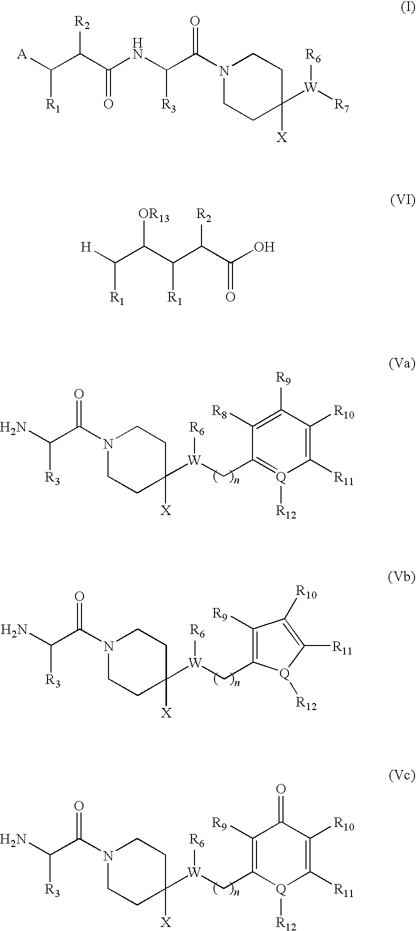
wherein, A, R₁, R₂, R₃, R₆, R₇, R₈, R₉, R₁₀, R₁₁, R₁₂, R₁₃, Q, W, X and n are the same as defined in claim 1.

7. An antibacterial composition comprising a therapeutically effective amount of the compound of formula (I) according to claim 1 or a pharmaceutically acceptable salt thereof.
